(19) United States
(12) Patent Application Publication
(10) Pub. No.: US 2023/0397872 A1
(43) Pub. Date: Dec. 14, 2023

(54) SYSTEMS AND METHODS FOR DETERMINING ELECTRODE-SKIN CONTACT USING SIGNAL UNDERSAMPLING

(71) Applicant: ZOLL Medical Corporation, Chelmsford, MA (US)

(72) Inventors: Nathan J. Berry Ann, Cranberry Township, PA (US); Philip E. Russell, Mt. Pleasant, PA (US); Earl E. Izydore, Wexford, PA (US)

(21) Appl. No.: 18/330,702

(22) Filed: Jun. 7, 2023

Related U.S. Application Data
(60) Provisional application No. 63/350,246, filed on Jun. 8, 2022.

(51) Int. Cl. A61B 5/282; A61N 1/04; A61B 5/0205

(52) U.S. Cl. CPC A61B 5/282; A61N 1/046; A61B 5/0205; A61B 5/0006

(57) ABSTRACT

A system for removing device signals from surface electrical signals received from an ambulatory patient worn device is provided. The system includes a wearable cardiac monitoring device configured to be continuously worn by a patient. The wearable cardiac monitoring device includes signal generation circuitry to provide a device-generated signal having a predetermined frequency; a skin-contacting surface to inject the device-generated signal into a skin of the patient; an electrode to acquire the surface electrical signal from the skin of the patient; and processing circuitry. The surface electrical signal includes a device-generated signal and a physiological signal generated by the patient. The processing circuitry samples the surface electrical signal from the patient, identifies, based on the predetermined frequency, aliasing content generated by the device-generated signal within the surface electrical signal, filters the aliasing content generated by the device-generated signal to isolate the physiological signal, and provides the physiological signal.

### CROSS-REFERENCE TO RELATED APPLICATIONS

This application claims the benefit of U.S. Provisional Patent Application 63/350,246 (filed 8 Jun. 2022), the entire disclosure of which is hereby incorporated by reference herein.

### BACKGROUND

The present disclosure is directed to sensors incorporated within a medical device for a variety of monitoring, diagnostic, and treatment purposes.

Heart failure, if left untreated, can lead to certain life-threatening arrhythmias. Both atrial and ventricular arrhythmias are common in patients with heart failure. One of the deadliest cardiac arrhythmias is ventricular fibrillation, which occurs when normal, regular electrical impulses are replaced by irregular and rapid impulses, causing the heart muscle to stop normal contractions. Because the victim has no perceptible warning of the impending fibrillation, death often occurs before the necessary medical assistance can arrive. Other cardiac arrhythmias can include excessively slow heart rates known as bradycardia or excessively fast heart rates known as tachycardia. Cardiac arrest can occur when a patient in which various arrhythmias of the heart, such as ventricular fibrillation, ventricular tachycardia, pulseless electrical activity (PEA), and asystole (heart stops all electrical activity), result in the heart providing insufficient levels of blood flow to the brain and other vital organs for the support of life. It is generally useful to monitor heart failure patients to assess heart failure symptoms early and provide interventional therapies as soon as possible.

Patients who are at risk of, have been hospitalized for, or otherwise are suffering from, adverse heart conditions can be prescribed a wearable cardiac monitoring and/or treatment device. In addition to the wearable device, the patient can also be given a battery charger and a set of rechargeable batteries. As the wearable device is generally prescribed for continuous or near-continuous use (for example, only to be removed when bathing), the patient wears the device during all daily activities such as walking, sitting, climbing stairs, resting or sleeping, and other similar daily activities. Maintaining continuous or near-continuous use of the device as prescribed can be beneficial for monitoring patient progress as well as providing treatment to the patient if needed.

### SUMMARY

In at least one example, a system for removing device signals from surface electrical signals received from an ambulatory patient worn device is provided. The system includes a wearable cardiac monitoring device configured to be continuously worn by a patient. The wearable cardiac monitoring device includes signal generation circuitry configured to provide at least one device-generated signal having a predetermined frequency; a skin-contacting surface configured to inject the at least one device-generated signal into a skin of the patient; at least one electrode configured to acquire at least one surface electrical signal from the skin of the patient, the at least one surface electrical signal including the at least one device-generated signal and at least one physiological signal generated by the patient; and processing circuitry different from the signal generation circuitry. The processing circuitry includes at least one processor configured to sample the at least one surface electrical signal from the patient, identify, based on the predetermined frequency, aliasing content generated by the at least one device-generated signal within the at least one surface electrical signal, filter the aliasing content generated by the at least one device-generated signal to isolate the at least one physiological signal, and provide the at least one physiological signal.

The system can include one or more of the following features. In the system, the processing circuitry and the signal generation circuitry can be asynchronous. A phase of the at least one device-generated signal provided by the signal generation circuitry can be arbitrary relative to a phase of the at least one device-generated signal within the at least one surface electrical signal. The predetermined frequency can be 100 Hz, 200 Hz, 300 Hz, 400 Hz, 500 Hz, 1 kHz, 2 kHz, 4 kHz, or 8 kHz. To sample can include to sample at a predetermined rate; and the predetermined frequency can be an integer multiple of the predetermined rate. The integer multiple can be 2, 3, 4, 5, or 6.

In the system, to sample can include to sample at a predetermined rate; the predetermined frequency can be an integer multiple of the predetermined rate; and the at least one processor can be further configured to control the signal generation circuitry to vary the predetermined frequency among a range of predetermined frequency values, determine a quality metric of the at least one surface electrical signal during provision of the at least one device-generated signal at each value of the range of predetermined frequency values, and identify the predetermined frequency value at which the quality metric meets at least one predetermined criterion. The range of predetermined frequency values can include one or more of 100 Hz, 200 Hz, 300 Hz, 400 Hz, 500 Hz, 1 kHz, 2 kHz, 4 kHz, or 8 kHz. The at least one predetermined criterion can include one or more of a threshold value or a relative quality metric. The quality metric can include one or more of a signal to noise ratio or an electrocardiogram (ECG) recognition metric.

In the system, the at least one processor can be further configured to isolate the at least one device-generated signal from the at least one surface electrical signal either periodically or continuously. The skin-contacting surface can be part of at least one of a therapy electrode, an ECG electrode, a combination therapy and ECG electrode, a dry/capacitive electrode, and a fabric electrode. To provide the at least one device-generated signal can include to modulate, by the signal generation circuitry, a base electrical signal at the predetermined frequency. The at least one processor can be configured to implement at least a portion of the signal generation circuitry in software. The at least one electrode can include a plurality of electrodes. The at least one surface electrical signal can include a plurality of surface electrical signals. To identify can include to identify the aliasing content generated by the at least one device-generated signal within each surface electrical signal of the plurality of surface electrical signals.

The system can include a clock circuit shared by the signal generation circuitry and the at least one processor. The clock circuit can be configured to provide a common reference point to the signal generation circuitry and the at least one processor.

The system can further include a network interface coupled to the at least one processor and to provide the at least one physiological signal can include to transmit the at least one physiological signal to a remote server distinct from the wearable cardiac monitoring device via the network interface. The at least one physiological signal can include an ECG signal.

In at least one example, a system for removing device signals from surface electrical signals received from an ambulatory patient worn device is provided. The system includes a wearable cardiac monitoring and treatment device configured to be continuously worn by a patient. The wearable cardiac monitoring and treatment device includes a garment wearable by the patient; signal generation circuitry configured to provide at least one device-generated signal having a predetermined frequency; at least one therapy electrode coupled to the garment and configured to discharge electrotherapy to a skin of the patient; at least one electrocardiogram (ECG) electrode coupled to the garment and configured to acquire at least one surface electrical signal from the skin of the patient, the at least one surface electrical signal comprising the at least one device-generated signal and at least one physiological signal generated by the patient; a skin-contacting surface configured to inject the at least one device-generated signal into the skin of the patient, the skin-contacting surface being a part of one or more of the at least one therapy electrode or the at least one ECG electrode; and processing circuitry coupled to the signal generation circuitry, the at least one therapy electrode, and the at least one ECG electrode. The processing circuitry includes at least one processor configured to sample the at least one surface electrical signal from the patient, identify, based on the predetermined frequency, aliasing content generated by the at least one device-generated signal within the at least one surface electrical signal, filter the aliasing content generated by the at least one device-generated signal to isolate the at least one physiological signal, and provide the at least one physiological signal.

The system can include one or more of the following features. In the system, the processing circuitry and the signal generation circuitry can be asynchronous. A phase of the at least one device-generated signal provided by the signal generation circuitry can be arbitrary relative to a phase of the at least one device-generated signal within the at least one surface electrical signal. The predetermined frequency can be 100 Hz, 200 Hz, 300 Hz, 400 Hz, 500 Hz, 1 kHz, 2 kHz, 4 kHz, or 8 kHz. To sample can include to sample at a predetermined rate and the predetermined frequency can be an integer multiple of the predetermined rate. The integer multiple can be 2, 3, 4, 5, or 6.

In the system, to sample can include to sample at a predetermined rate; the predetermined frequency can be an integer multiple of the predetermined rate; and the at least one processor can be further configured to control the signal generation circuitry to vary the predetermined frequency among a range of predetermined frequency values, determine a quality metric of the at least one surface electrical signal during provision of the at least one device-generated signal at each value of the range of predetermined frequency values, and identify a predetermined frequency value at which the quality metric meets at least one predetermined criterion. The range of predetermined frequency values can include one or more of 100 Hz, 200 Hz, 300 Hz, 400 Hz, 500 Hz, 1 kHz, 2 kHz, 4 kHz, or 8 kHz. The at least one predetermined criterion can include one or more of a threshold value or a relative quality metric. The quality metric can include one or more of a signal to noise ratio or an electrocardiogram (ECG) recognition metric. The at least one processor can be further configured to isolate the at least one device-generated signal from the at least one surface electrical signal either periodically or continuously.

In the system, to provide the at least one device-generated signal can include to modulate, by the signal generation circuitry, a base electrical signal at the predetermined frequency. The at least on processor can be configured to implement at least a portion of the signal generation circuitry in software. In the system, the at least one ECG electrode can include a plurality of ECG electrodes; the at least one surface electrical signal can include a plurality of surface electrical signals; and to identify can include to identify the aliasing content generated by the at least one device-generated signal within each surface electrical signal of the plurality of surface electrical signals.

The system can further include a clock circuit shared by the signal generation circuitry and the at least one processor. The clock circuit can be configured to provide a common reference point to the signal generation circuitry and the at least one processor.

The system can further include a network interface coupled to the at least one processor and to provide the at least one physiological signal can include to transmit the at least one physiological signal to a remote server distinct from the wearable cardiac monitoring and treatment device via the network interface. The at least one physiological signal can include an ECG signal.

In at least one example, a method for removing device signals from surface electrical signals received from an ambulatory patient worn device is provided. The method includes generating at least one device-generated electrical signal at a predetermined frequency; injecting the at least one device-generated signal into a skin of a patient; sampling at least one surface electrical signal from the patient at a predetermined rate, the at least one surface electrical signal comprising the at least one device-generated signal and at least one physiological signal generated by the patient, wherein a phase of the at least one device-generated signal as included in the at least one surface electrical signal can be arbitrary relative to a phase of the at least one device-generated signal as injected into the skin of the patient; identifying, based on the predetermined frequency, aliasing content generated by the at least one device-generated electrical signal within the at least one surface electrical signal; filtering the aliasing content generated by the at least one device-generated electrical signal to isolate the at least one physiological signal; and providing the at least one physiological signal.

The method can include one or more of the following features. In the method, generating the at least one device-generated electrical signal can include generating the at least one device-generated electrical signal at a predetermined frequency of 100 Hz, 200 Hz, 300 Hz, 400 Hz, 500 Hz, 1 kHz, 2 kHz, 4 kHz, or 8 kHz. Generating the at least one device-generated electrical signal can include generating the at least one device-generated electrical signal at a predetermined frequency that is an integer multiple of the predetermined rate. Generating the at least one device-generated electrical signal can include generating the at least one device-generated electrical signal at a predetermined frequency that is 2, 3, 4, 5, or 6 times the predetermined rate.

The method can further include varying the predetermined frequency among a range of predetermined frequency values; determining a quality metric of the at least one surface electrical signal during provision of the at least one device-generated electrical signal at each value of the range of predetermined frequency values; and identifying a predetermined frequency value at which the quality metric meets at least one predetermined criterion. In the method, varying the predetermined frequency can include varying the predetermined frequency among 100 Hz, 200 Hz, 300 Hz, 400 Hz, 500 Hz, 1 kHz, 2 kHz, 4 kHz, and 8 kHz. Identifying the predetermined frequency value can include evaluating one or more of a threshold value or a relative quality metric. Determining the quality metric can include determining one or more of a signal to noise ratio or an electrocardiogram (ECG) recognition metric. The method can further include isolating the at least one device-generated electrical signal from the at least one surface electrical signal either periodically or continuously. Generating the at least one device-generated electrical signal can include modulating a base electrical signal at the predetermined frequency.

In the method, sampling the at least one surface electrical signal can include sampling a plurality of surface electrical signals and identifying the aliasing content can include identifying the aliasing content generated by the electrical signal within each surface electrical signal of the plurality of surface electrical signals. The method can further include generating a clock signal to provide a common reference point for generating the at least one device-generated electrical signal at the predetermined frequency and sampling the at least one surface electrical signal from the patient at the predetermined rate. In the method, providing the at least one physiological signal can include transmitting the at least one physiological signal to a remote server. Sampling the at least one surface electrical signal can include sampling at least one surface electrical signal including an ECG signal generated by the patient.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples and are incorporated in and constitute a part of this specification but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIGS. 1A and 1B illustrate sample sensor arrangements, in accordance with an example of the present disclosure.

FIG. 2A illustrates a sample wearable cardiac device including sensors having a broadcast device-generated signal, in accordance with an example of the present disclosure.

FIG. 2B illustrates another sample wearable cardiac device including sensors having a broadcast device-generated signal, in accordance with an example of the present disclosure.

FIG. 2C illustrates a sample wearable cardiac device including sensors having a localized device-generated signal, in accordance with an example of the present disclosure.

FIG. 3A illustrates a sample therapy electrode assembly including circuitry for outputting a device-generated signal, in accordance with an example of the present disclosure.

FIG. 3B illustrates another sample therapy electrode assembly including circuitry for outputting a device-generated signal, in accordance with an example of the present disclosure.

FIGS. 4A to 4C illustrate various sample sensor assemblies, in accordance with examples of the present disclosure.

FIG. 5 illustrates a sample arrangement including multiple sensor assemblies arranged into a sample sensor pair, in accordance with an example of the present disclosure.

FIG. 6 illustrates a schematic view of a sample controller for a wearable medical device, in accordance with an example of the present disclosure.

FIG. 7A illustrates a sample conditioning and filtering circuit for isolating aliasing context within surface electrical signals, in accordance with an example of the present disclosure.

FIGS. 7B and 7C illustrate sample circuit diagrams for various components included within the circuit shown in FIG. 7A, in accordance with an example of the present disclosure.

FIG. 8 illustrates a sample process flow for determining and isolating aliasing content within surface electrical signals, in accordance with an example of the present disclosure.

FIG. 9A illustrates a more detailed sample process flow for determining and isolating aliasing content within surface electrical signals, in accordance with an example of the present disclosure.

FIG. 9B illustrates a sample process flow for dynamically determining a frequency for a device-generated signal, in accordance with an example of the present disclosure.

FIG. 10A illustrates graphs representing sample surface electrical signals, in accordance with an example of the present disclosure.

FIG. 10B illustrates a graph representing a second example of surface electrical signals, in accordance with an example of the present disclosure.

FIGS. 10C-10F illustrate sets of graphs representing experimental surface electrical signals and conditioned signals derived therefrom.

FIGS. 11A-11D illustrate sample ambulatory medical devices that may be prescribed to a heart failure patient, in accordance with examples of the present disclosure.

### DETAILED DESCRIPTION

Given that wearable monitoring devices are prescribed for continuous or near-continuous use, there is a need for systems, devices and methods that accurately and reliably monitor electrode skin contact. As such, this disclosure relates to systems, devices, and methods for determining electrode skin contact in long term continuous monitoring ECG and/or bioimpedance devices. Systems, devices, and methods that monitor a quality of electrode skin contact can be beneficial in such devices to improve reliability of the ECG or bioimpedance data. For example, arrhythmia monitoring systems (AMS), heart failure management systems (HFMS), mobile cardiac telemetry (MCT) systems, extended Holter (EH) systems, continuous event monitoring (CEMS) systems, and wearable cardioverter defibrillator systems (WCDs) provide long term and continuous ECG and/or bioimpedance monitoring. In all such systems, it is desirable for the devices to be able to determine whether electrode skin contact is properly established such that physiological signals received therefrom are within an expected operating range and/or are have minimal noise attributes, as described in further detail below. In the case of WCDs, the devices also additionally provide therapeutic electrical shocks to the patient. In such devices and systems, there is a need to improve the ability to reliably provide instantaneous or nearly instantaneous information about the quality of the electrode skin contact with the patient. In examples, the techniques for determining the quality of electrode skin contact involve sending electrical signals (for example, injecting electrical signals) through ECG electrodes. In such examples, it is desirable for associated processing circuitry and/or software implemented on corresponding microcontrollers to reliably separate device-generated signals from body surface electrical signals. In examples, wearable monitoring devices as described herein can perform bioimpedance spectroscopy or bioimpedance analysis on the patient. For example, such bioimpedance spectroscopy analysis can include single-frequency analysis, including measurement of body impedance at a predetermined frequency, such as 48 kHz, or multi-frequency bioimpedance spectroscopy, for example, providing current to the body at multiple frequencies, such as 35 Hz to 100 Hz, and measuring the impedance at each frequency.

Example wearable cardiac monitoring and/or treatment systems as disclosed herein are directed to providing for isolation and removal of a device-generated signal from a surface electrical signal, for example, improving the quality of a sensed physiological signal of a patient. By implementing an undersampling process as described herein, aliasing content within the surface electric signal caused by the device-generated signal can be isolated and removed, thereby providing for a filtered and conditioned physiological signal for further processing. In examples, the systems, devices, and techniques described herein facilitate continuous, noise-free electrode fall off or electrode lead off detection.

In a wearable cardiac monitoring system, the quality of contacts between a sensing electrode and the patient skin is often monitored for any potential “fall off” or “lead off” events. For example, such events include scenarios where one or more contact sensors, such as ECG electrodes, are not making proper contact with the skin of the patient. In this regard, such proper contact with the skin of the patient results in physiological signals, such as ECG signals, bioimpedance signals, or other such skin surface electrical signals, that are within design specifications and constitute an acceptable operating range. For example, such fall off or lead off events can be brief, when a patient is engaged in physical activity or physical movement that may temporarily or permanently cause the one or more sensors to not make proper contact with the skin of the patient, or cause the generated physiological signals to fall outside the acceptable operating range. For example, a patient may bend down from a standing position causing one or more ECG electrodes to briefly separate from the patient's skin. In another scenario, the one or more ECG electrodes may remain in contact with the patient's skin but the electrode may briefly not be applied with the appropriate pressure against the skin of the patient, for example, the applied pressure may be less than a predetermined design range such as 0.45 psi to 0.65 psi. In examples, fall off events may last for a longer period of time, such as when a patient incorrectly wears, applies, or attaches ECG electrodes against the patient's skin. For instance, when assembling a WCD, a patient may insert ECG or therapy electrodes incorrectly such that the contact surface of the electrode is oriented away from the patient's skin. In such scenarios, the systems, devices, and techniques described herein can improve reliability of the underlying sensor data.

In some examples, a device-generated signal such as a fall off signal is injected into the patient's body and measured at one or more sensing electrodes. Based upon various characteristics of the device-generated signal as received at each sensing electrode, the quality of contact between the sensing electrode and the patient skin can be determined. The device-generated signal can be applied to the patient's skin continuously or substantially continuously to enable ECG falloff detection. For example, the device-generated signal can be applied to the patient's skin continuously while the device is worn by the patient. For example, the device-generated signal can be applied to the patient's skin continuously while the device is worn by the patient and operational for its primary purpose, for example, a wearable cardiac monitoring device is operational when it is processing and/or detecting ECG and/or bioimpedance signals derived via ECG electrodes. For example, the device-generated signal can be applied to the patient's skin continuously while the device is worn by the patient and operational for its primary purpose, for example, a wearable cardiac monitoring and treatment device is operational when it is processing and/or detecting ECG and/or bioimpedance signals derived via ECG electrodes and able to provide therapeutic shocks via therapy electrodes on detecting life-threatening arrhythmias in the detected ECG signals. The device-generated signal is sensed by the ECG electrodes, and, in implementations herein, the amplitude of the device-generated signal can be much larger than the amplitude of the ECG signal. For these reasons, the device-generated signal can be a source of ECG noise.

In example implementations, the device-generated signal is produced based on a clock frequency. In examples, the clock frequency is selected as a predetermined integer multiple of the sampling rate of the underlying surface electrical signal. For example, the clock frequency can be selected to be an integer multiple of two, three, four, five, six, seven, eight, and so on of the analog-to digital converter (ADC) sampling rate. In this regard, the ECG ADCs intentionally undersample the received device-generated signal, aliasing it to frequencies that can be subsequently removed from the ECG signal by software filters, so that it does not appear as noise in the ECG waveform.

As noted above, in examples, an amplitude of the device-generated signal can be larger than the physiological signal sensed for the patient. For this reason, the device-generated signal can be a source of noise within a surface electrical signal if not isolated and removed from the surface electrical signal thereby resulting in the appropriate physiological signal. In a wearable cardiac monitoring system, noise and similar signal characteristics can degrade overall signal quality. For example, excess noise caused by a device-generated signal as described herein can degrade the quality of the physiological signal being sensed. With a degraded physiological signal, a medical device controller can require additional time to process the signal and/or output an incorrect determination when determining whether a patient is experiencing a cardiac arrhythmia. The systems and methods as described herein provide for an undersampling process that can be used to efficiently identify and remove aliasing content caused by the device-generated signal within surface electrical signals as detected by, for example, sensing electrodes included within a wearable cardiac monitoring system.

A wearable cardiac device as described herein can include signal generation circuitry configured to provide at least one device-generated signal at a predetermined frequency to be injected into the patient's body. For example, the device-generated injected signal includes a current produced by a current generator. For instance, the current generator can provide electrical signals (for example, injection current) to the skin of the user via the ECG or body impedance electrode. In examples, the current supplied can be at a predetermined single frequency or a current supplied at multiple frequencies. For example, the current generator can be used to provide the fall off current and/or a body impedance current in accordance with implementations described herein. In such cases where the current generator is providing body impedance current, the current generator can supply a current at multiple frequencies, for example, provide the injection current as multiple subsequent currents at different frequencies, for example, a sweep injection current that is configured to sweep through a range of frequencies such as 1 kHz to 200 MHz, 10 MHz to 200 MHz, or 100 MHz to 300 MHz. In some cases, the current generator can provide a current at multiple frequencies in the form of a broadband injection current over frequencies of around 1 kHz to 200 MHz, around 10 MHz to 200 MHz, or around 100 MHz to 300 MHz. The wearable cardiac device can include one or more sensing electrodes configured to sense surface electrical signals including physiological signals and device-generated signals as described herein. A processing device is further configured to sample the at least one surface electrical signal from the patient at a predetermined sampling rate and identify, based on the predetermined frequency of the device-generated signal, aliasing content generated by the device-generated signal within the at least one surface electrical signal. The processing device can further filter the aliasing content from the surface electrical signal to isolate the at least one physiological signal for further processing. In certain examples, the predetermined sampling rate can be selected such that the predetermined frequency of the device-generated signal is an integer multiple of the predetermined rate of sampling. In such an example, the processor can quickly and efficiently identify the aliasing content generated by the device-generated signal within the at least one surface electrical signal and filter the aliasing content to provide a high quality physiological signal for further processing.

These examples, and various other similar examples that benefit from the techniques, processes, and approaches as provided herein, are described in additional detail below.

A patient having an elevated risk of sudden cardiac death, unexplained syncope, prior symptoms of heart failure, an ejection fraction of less than 45%, less than 35%, or other such predetermined threshold deemed of concern by a physician, and other similar patients in a state of degraded cardiac health can be prescribed specialized cardiac monitoring and treatment devices, such as a mobile cardiac telemetry (MCT) device, a wearable cardioverter-defibrillator (WCD), and/or an hospital wearable defibrillator (HWD). Such medical devices can benefit from the incorporation of, or interoperation with, an adjustable garment.

For example, as described in greater detail below, a specific scenario is now illustrated. In this scenario, a continuous square wave device-generated signal can be applied to the patient using rear therapy electrodes disposed in a garment of a wearable cardioverter defibrillator. While in this scenario, the signal is applied via the rear therapy electrode, in other implementations, the signal can be applied via a front therapy electrode, an ECG electrode and/or a dedicated device-generated signal injection electrode. For example, a processor is configured to produce the square wave output at a predetermined frequency F1×n, where F1 is the frequency at which the signal processing circuitry associated with the ECG electrodes is configured to sample the ECG data. For example, the processor can be disposed in a belt node housing, or within housing of the rear therapy electrode, or within housing of an ECG electrode, or within housing of a dedicated device-generated signal injection electrode. For example, the device-generated signal can be a 8 kHz, 40 mV peak to peak signal. For example, the device-generated signal can have a preconfigured amplitude in a range from about 10 mV to about 100 mV peak to peak, or from about 100 mV to about 500 mV peak to peak. When an ECG electrode is in contact with the patient's skin, the ECG electrode can sense the device-generated signal injected into the patient at some amplitude, for example, less than or equal to 40 mV pp (depending on the initial preconfigured amplitude). In these examples, the initial phase of the device-generated signal is not relevant. For example, the signal processing circuitry associated with the ECG electrodes may not be synchronized to the clock of the signal generation circuit that produced the device-generated signal. In this regard, the signal processing circuitry associated with the ECG electrodes picks up that the device-generated signal is in an indifferent phase and further processes the signal as described below. As noted, the amplitude of the device-generated signal can be greater than the ECG signal itself and as such must be removed with post-processing software and/or hardware filters.

In some implementations (with digital ECG electrodes), each ECG sensor can include an ADC that samples the ECG signal (along with the device-generated signal) at, say, 2 kHz. By sampling the 8 kHz signal at 2 kHz, the system is intentionally undersampling the device-generated signal in order to intentionally alias the device-generated signal.

In such digital ECG electrode implementations, the sampled ECG data can be transmitted from each ECG electrode to a belt node processor or other processor downstream where the signal is filtered and digitized to produce, for example, 400 sample-per-second (SPS) data with an analog bandwidth of approximately 0.5 Hz to 165 Hz. In this regard, because the 8 kHz is an integer multiple of the 2 kHz sample rate, the in-band alias is positioned at 0 Hz (DC). As such, the software filtering applied to the ECG signal at the belt node processor can remove the alias content, to maintain a low-noise ECG signal.

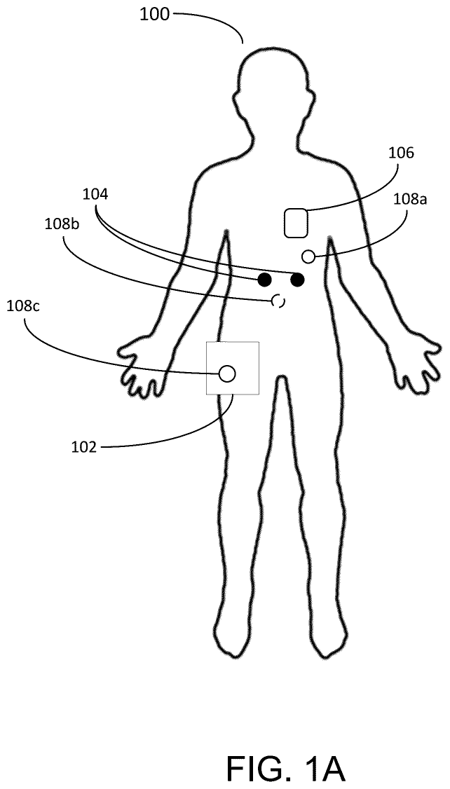

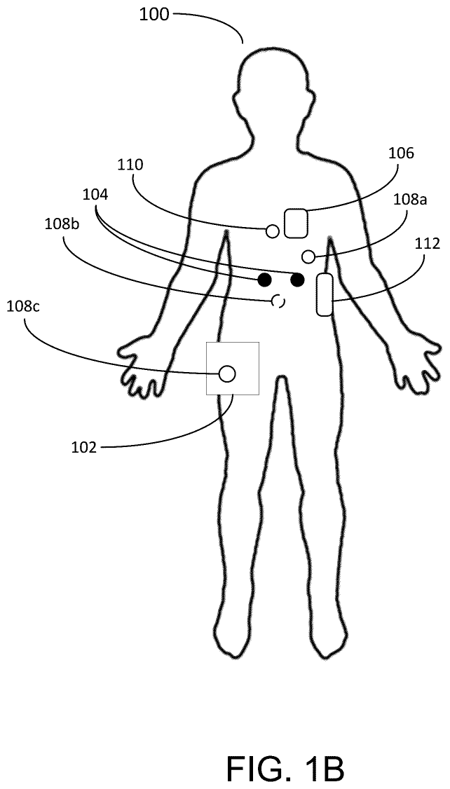

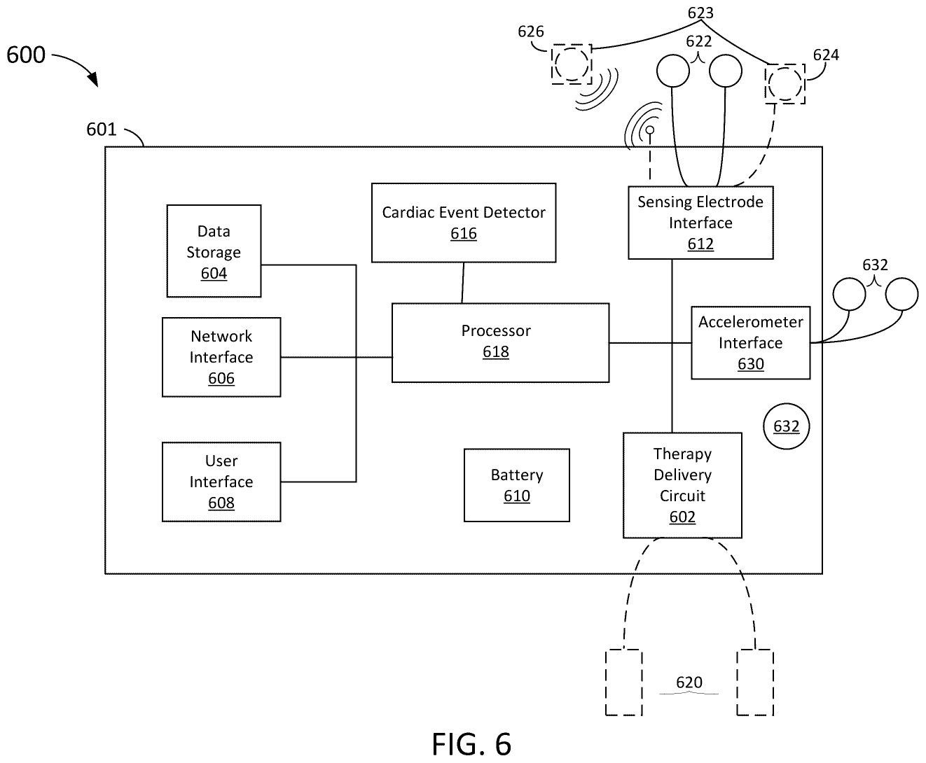

FIGS. 1A and 1B illustrate various examples of a patient 100 wearing medical devices that include one or more sensors (for example, sensing electrodes, accelerometers, audio and/or vibrational sensors, radio frequency (RF) sensors, and stretch or pressure sensors). As shown in FIG. 1A, the patient 100 can be prescribed an ambulatory medical device such as a WCD (or, for an in-hospital patient, an HWD). The WCD can include a controller 102 that is operably connected to one or more sensing electrodes 104 and therapy electrodes 106. Additional details of examples of the controller 102, sensing electrodes 104, and therapy electrodes 106 can be found in the discussion of FIG. 6 below.

The WCD can also include one or more accelerometers or other motion sensors. As shown in FIG. 1A, the WCD can include three accelerometers 108a, 108b, and 108c (collectively referred to as accelerometers 108) positioned at various places on the body of patient 100. For example, accelerometer 108a can be positioned on the front of the chest of the patient 100, the accelerometer 108b can be positioned on the back of the patient 100, and the accelerometer 108c can be integrated into the controller 102. Each of the accelerometers 108 can be configured to measure movement associated with the patient 100 and to output an electrical signal indicating a direction and magnitude of the movement of the patient 100.

It should be noted that the number and arrangement of the accelerometers 108 as shown in FIG. 1A is by way of example only. In certain implementations, the number and position of the accelerometers 108 can vary. Additionally, when included in a device such as a WCD, one or more of the accelerometers 108 can be integrated into components of the WCD. For example, as noted above, the accelerometer 108c can be integrated into the controller 102 of the WCD. Similarly, one or more of accelerometers 108a and 108b can be integrated into one or more components of the WCD. For example, the front accelerometer 108a can be integrated into the therapy electrode 106, which is operably connected to the controller 102 and configured to provide a therapeutic shock to the patient 100. In some implementations, the accelerometer 108a can be integrated into one of the sensing electrodes 104, which are configured to measure electrical signals produced by the patient 100 that are indicative of cardiac activity of the patient 100. Similarly, accelerometer 108b can be integrated into one or more components of the WCD such as a connection node, at least one sensing electrode 104, the therapy electrode 106, and other similar components of the WCD as described herein. Alternatively or additionally, the one or more accelerometers 108 can be distinct components of the WCD.

In HWD implementations, the accelerometers can be integrated into one or more adhesive ECG sensing and/or therapy electrode patches. For example, a first accelerometer can be integrated into a first adhesive ECG sensing and/or therapy electrode patch and a second accelerometer can be integrated into a second adhesive ECG sensing and/or therapy electrode patch. Additional accelerometers can be disposed within a controller (similar to the controller 102 of a WCD) associated with the HWD.

In addition to accelerometers associated with a WCD as described above in regard to FIG. 1A, a patient such as the patient 100 can also wear additional sensors. As shown in FIG. 1B, the patient 100 can wear a vibrational sensor 110 that is configured to record bio-vibrational signals of the patient 100. For example, the vibrational sensor 110 can be configured to detect vibrations of the patient 100 that are associated with, for example, heart and lung activity. In certain implementations, the vibrational sensor 110 can be configured to detect cardiovibrational values including any one or all of S1, S2, S3, and S4. From these cardiovibrational values, certain heart vibration metrics or combinational metrics may be calculated, including any one or more of electromechanical activation time (EMAT), left ventricular systolic time (LVST), or percentage of left ventricular systolic time (% LVST). In some examples, the vibrational sensor 110 can be configured to detect vibrations from the cardiac system of the patient 100 and provide an output signal responsive to the detected cardiovibrational values. The vibrational sensor 110 can also include a multi-channel accelerometer, for example, a three-channel accelerometer configured to sense movement in each of three orthogonal axes such that patient movement/body position can be detected and correlated to detected cardiovibrational values. The vibrational sensor 110 can transmit information descriptive of the cardiovibrational values to, for example, a sensor interface for subsequent analysis as described below.

Additionally, the patient 100 can wear an RF sensor 112. For example, the RF sensor 112 can be configured to use RF-based techniques to assess fluid levels and accumulation in body tissue of the patient 100. For instance, the RF sensor 112 can be configured to measure fluid content in the lungs, typically for diagnosis and follow-up of pulmonary edema or lung congestion in heart failure patients. Similarly, the RF sensor can be configured to measure thoracic fluid content for the patient 100. In certain implementations, the RF sensor 112 can include one or more antennas configured to direct radio frequency waves through tissue of the patient 100 and measure output radio frequency signals in response to the waves that have passed through the tissue. In certain implementations, the output radio frequency signals include measured characteristics, parameters and/or attributes indicative of a fluid level in the tissue. The RF sensor 112 can transmit information descriptive of the tissue fluid levels to a sensor interface for subsequent analysis as described below.

It should be noted that the placement and number of sensors as shown in FIGS. 1A and 1B are shown by way of example only. In actual implementation of the medical devices as described herein, the number and position of the sensors can vary based upon the type of patient monitoring and/or treatment to be performed and other various factors.

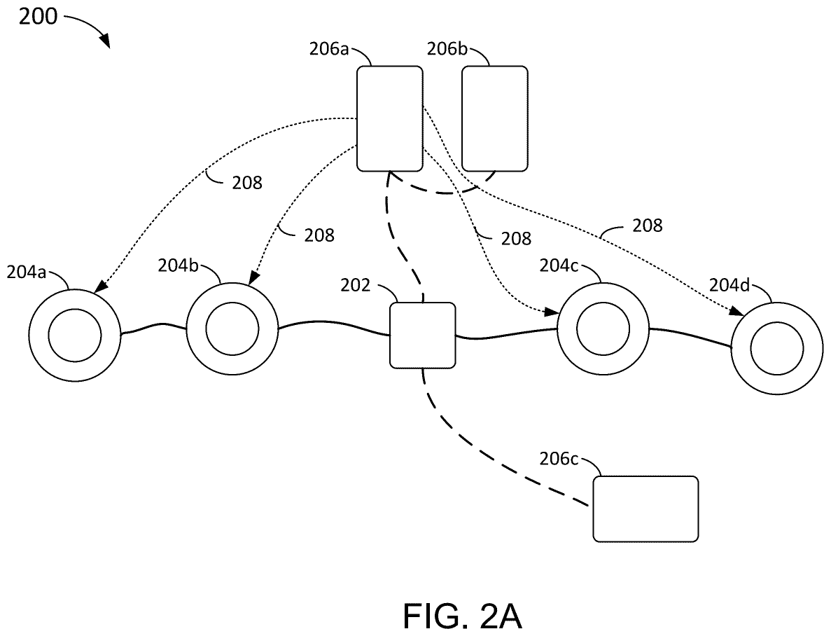

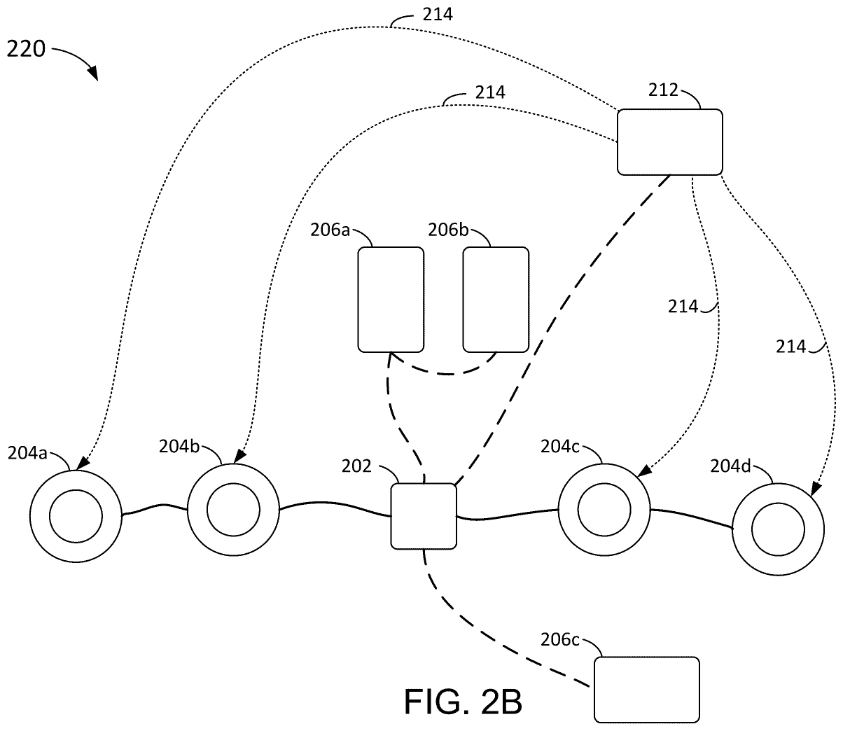

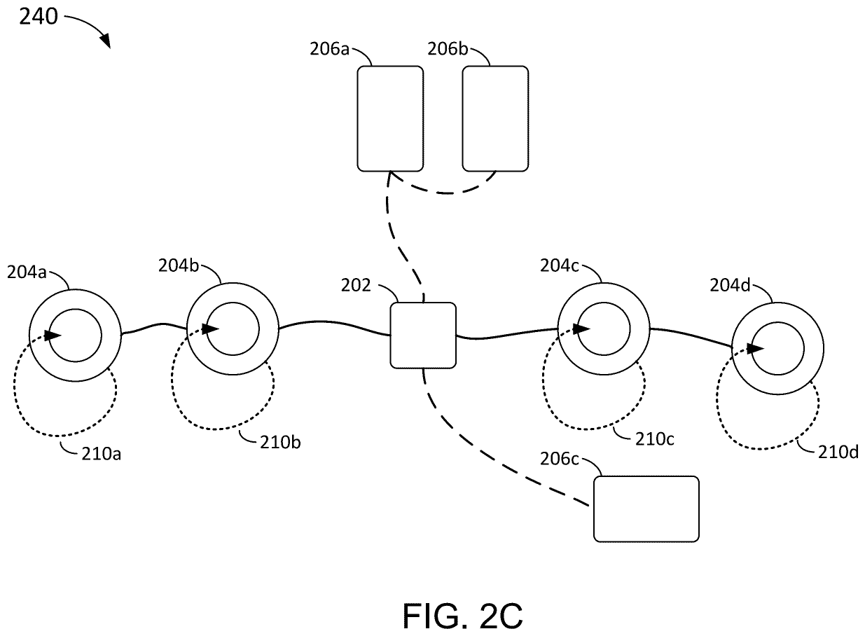

FIG. 2A illustrates system 200 including the components of a wearable medical device including, for example, a WCD as described herein. As shown in FIG. 2A, the components of the wearable medical device can be operably connected to a belt node 202 including, for example, a belt node or other similar node configured to both receive signals from and transmit signals to the components of the wearable medical device. For example, as shown in FIG. 2A, the belt node 202 can be operably connected to a set of sensing electrodes 204a to 204d. In some implementations, the belt node 202 comprises a housing configured to include signal generating circuitry including processors for generating the device-generated signal as described herein. Alternatively or additionally, the belt node 202 houses signal processing circuitry including processors for processing the ECG data from the ECG electrodes. Alternatively or additionally, the belt node 202 houses gel filling circuitry configured to cause gel to be deployed on the patient's skin prior to delivering an electric shock to the patient via the therapy electrodes. Alternatively or additionally, the belt node 202 houses vibration circuitry with a vibration motor that operates under control of a processor to deliver vibration alerts to the patient. In certain implementations, if the wearable medical device includes treatment functionality, a set of therapy electrodes 206a to 206c can also be operably coupled to the belt node 202. In some examples, the belt node 202 can be further coupled to a controller or another processing device configured to receive signals from the belt node 202, process the signals received from the belt node 202, and send control signals back to the belt node 202. As such, the belt node 202 can be configured to operate as an ECG acquisition node or box that is configured to determine an ECG signal of the patient based on a received surface electrical signals as received from each of the sensing electrodes 204a to 204d as described herein.

As further shown in FIG. 2A, one or more of the therapy electrodes 206a to 206c can be configured to include circuitry configured to generate and output a device-generated signal. For example, this signal can be generated at a predetermined frequency and broadcast or otherwise output by at least one of the therapy electrodes 206a to 206c into the patient's body. As shown in FIG. 2A, this signal is output by therapy electrode 206a. However, this is shown by way of example only.

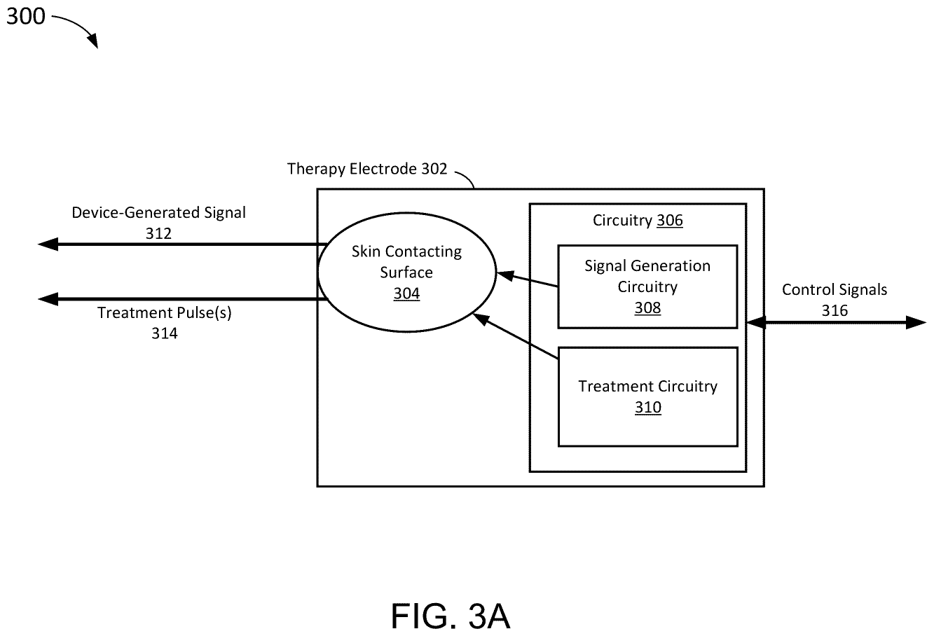

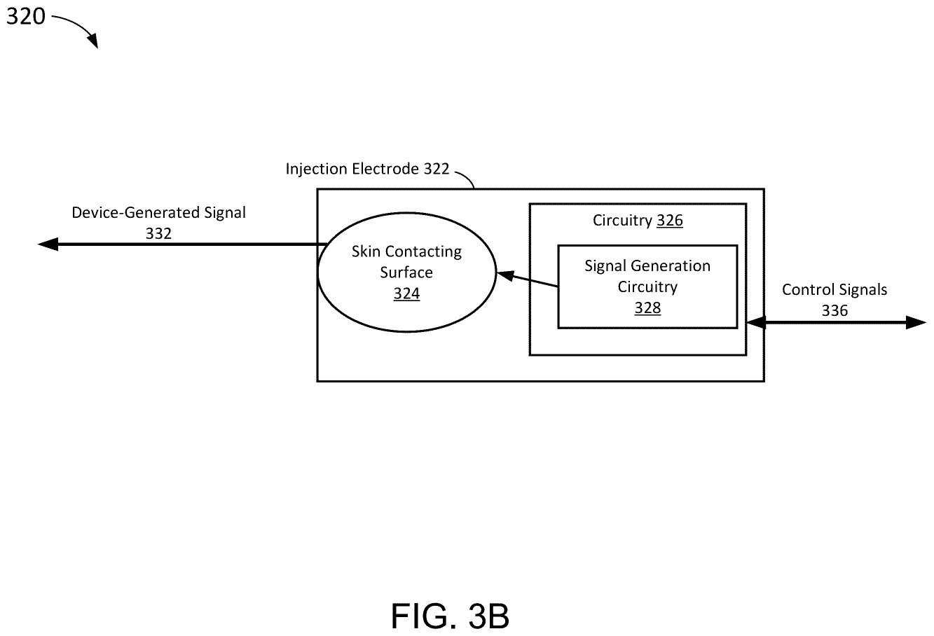

More specifically, as shown in FIG. 2A, the therapy electrode 206a is configured to output a device-generated signal 208. The device-generated signal 208 transmitted by the therapy electrode 206a can be picked up by one or more of the sensing electrodes 204a to 204d. In certain implementations, as shown in FIG. 2A and described in greater detail in the discussion of FIG. 3A, each of the therapy electrodes 206a to 206c can include a skin contact surface configured to be in physical contact with the skin of the patient. Each therapy electrode 206a to 206c can also include signal generation circuitry configured to generate the device-generated signal 208 such that the signal is output into the patient's body via the skin contacting surface. Each of sensing electrodes 204a to 204d can be configured to receive or otherwise sense a surface electrical signal that includes at least a portion of the device-generated signal 208.

In addition to the example shown in FIG. 2A, the device-generated signal can be injected into the patient's body via a dedicated current injection electrode. For instance, as shown in system 220 of FIG. 2B, a current injection electrode 212 can be configured to include circuitry to generate and output a device-generated signal 214. The device-generated signal 214 transmitted by the current injection electrode 212 can be picked up by the one or more of the sensing electrodes 204a to 204d. In certain implementations, as shown in FIG. 2B and described in greater detail in the discussion of FIG. 3B, the current injection electrode 212 can include a skin contact surface configured to be in physical contact with the skin of the patient. The current injection electrode 212 can also include signal generation circuitry configured to generate the device-generated signal 214 such that the signal is output into the patient's body via the skin contacting surface. Each of sensing electrodes 204a to 204d can be configured to receive or otherwise sense a surface electrical signal that includes at least a portion of the device-generated signal 214.

As shown in FIGS. 2A and 2B, the device-generated signal can be injected into the patient's body via one or more of the therapy electrodes 206a to 206c and/or a current injection electrode 212. However, in certain implementations, one or more of the sensing electrodes 204a to 204d can be configured to output the device-generated signal as well as to sense or otherwise receive the device-generated signal from the patient's body. For example, as shown in system 240 of FIG. 2C, each of the sensing electrodes 204a to 204d can be configured to include circuitry to generate and output a localized version of the device-generated signal. As described herein, by providing each individual sensing electrode with the circuitry to generate and output a localized version of the device-generated signal, overall redundancy in the system can be improved as sensing electrodes can further detect a localized version of the device-generated signals from other sensing electrodes in the event of a malfunction of an individual electrode's ability to generate a localized version of the device-generated signal.

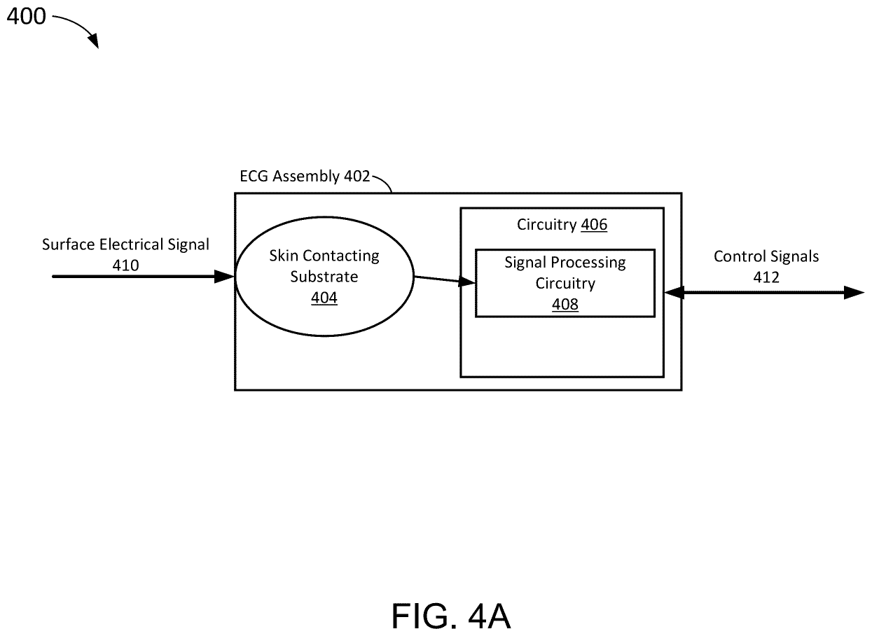

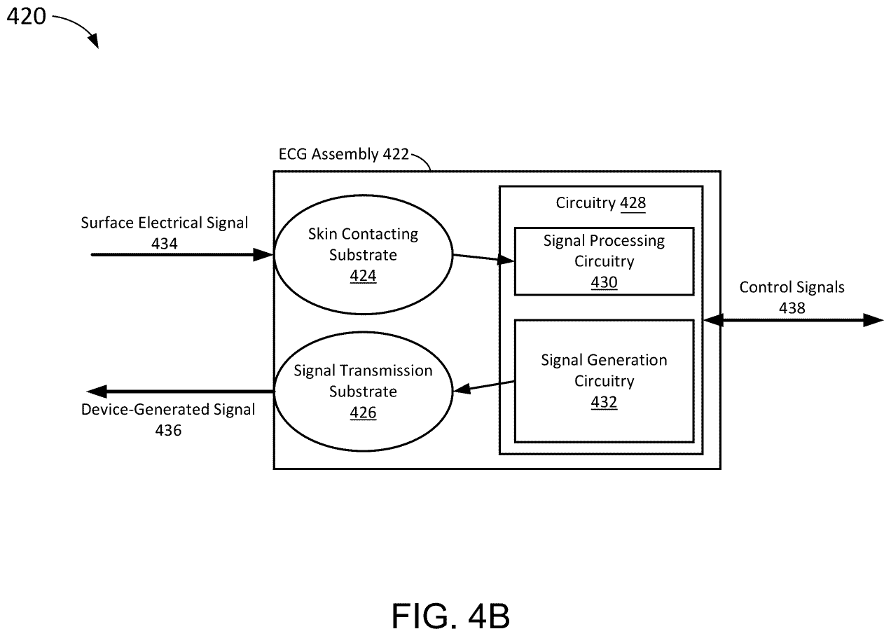

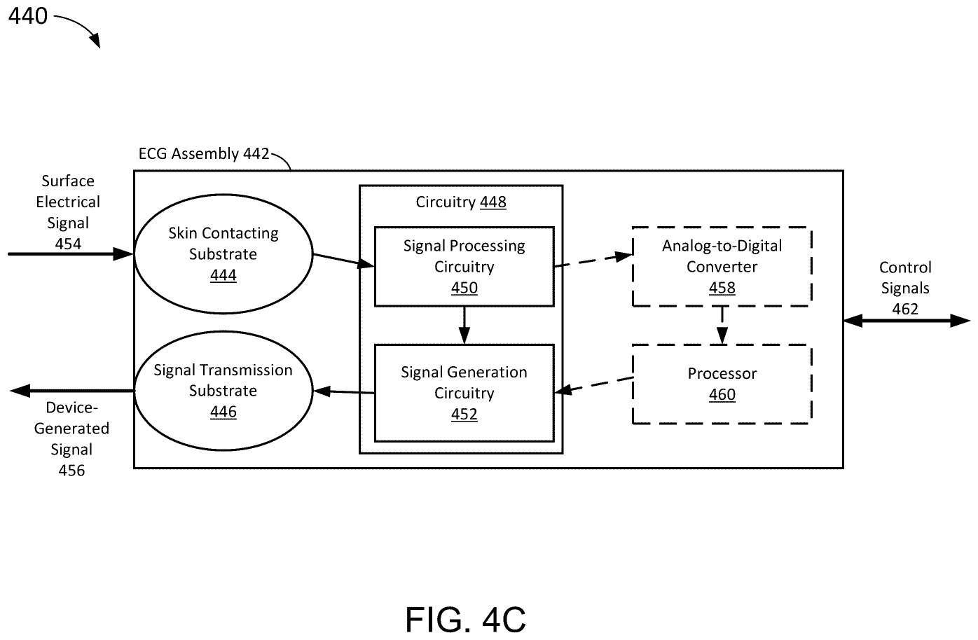

More specifically, as shown in FIG. 2C and described in greater detail in the discussion of FIGS. 4B and 4C, each of the sensing electrodes 204a to 204d can include an ECG electrode substrate configured to be in physical contact with the skin of the patient and sense, detect, or otherwise receive one or more surface electrical signals from the patient. Additionally, each of the sensing electrodes 204a to 204d can further include a signal transmission substrate proximate to the ECG electrode substrate and also configured to be in physical contact with the skin of the patient. The signal transmission substrate can be configured to provide a localized version of the device-generated signal to the patient's body. For example, as shown in FIG. 2C, sensing electrode 204a can be configured to transmit and receive localized device-generated signal 210a. Similarly, sensing electrode 204b can be configured to transmit and receive localized device-generated signal 210b, sensing electrode 204c can be configured to transmit and receive localized device-generated signal 210c, and sensing electrode 204d can be configured to transmit and receive device-generated signal 210d.

In some examples, each of the device-generated signals 210a to 210d can be synchronized and have the same output frequency. In such an example, the local versions of the device-generated signals 210a to 210d do not interfere with the other sensing electrodes 204a to 204d. In certain implementations, such an example can be used to provide for redundancy. For example, if sensing electrode 204a is incapable of outputting local device-generated signal 210a, the sensing electrode may be able to receive one or more of device-generated signals 210b to 210d. If each of the device-generated signals 210a to 210d have the same output frequency as described herein, a controller or other similar processing device can use the device-generated signal as received at any of the sensing electrodes 204a to 204d to determine the quality of the electrode-skin interface (for example, whether the sensing electrode is in proper contact with the patient's skin as described above) as well as removing aliasing content caused by the device-generated signals regardless of which sensing electrode generates and output the device-generated signal. As noted previously, a determination of when one or more ECG electrodes are not making proper contact with skin of the patient can be based on whether the associated signal processing circuitry is able to generate physiological signals that are within an acceptable operating range (for example, produce signals that are within a 0.5 mV to 10.0 mV, combined with a DC component of up to ±300 mV to 500 mV). For example, fall off events involving improper electrode contact can be brief or permanent, and cause the one or more sensors to not make proper contact with the skin of the patient, or cause the generated physiological signals to fall outside the acceptable operating range. For example, the one or more ECG electrodes may remain in contact with the patient's skin, but the electrode may briefly not be applied with the appropriate pressure against the skin of the patient, for example, the applied pressure may be less than a predetermined design range such as 0.45 psi to 0.65 psi.

FIG. 3A illustrates a system 300 including a more detailed schematic view of a therapy electrode such as one of therapy electrodes 206a to 206c as shown in FIG. 2A and described above. For example, as shown in FIG. 3A, a therapy electrode 302 can include a skin contacting surface 304 and circuitry 306. As further described herein, the circuitry 306 can include signal generation circuitry 308 (for example, current generator circuitry configured to provide currents at one or more predetermined frequencies) and treatment circuitry 310. For example, the current generator circuitry 308 can generate a sinusoidal wave, a square wave, a rectangular wave, a triangular wave, a sawtooth wave, a pulse wave, a chirp signal, or other such base as can be output via a function generator. For example, such a current generator can incorporate a precision waveform generator integrated circuit that is configured to generate output waveforms such as sine, square and triangular output waveforms, with a minimum number of external components or adjustments. The circuit includes an operating frequency range that can be selected over eight decades of frequency, from 0.001 Hz to 300 kHz, by the correct choice of the external R-C components. A microprocessor with integrated timing peripherals can also be used. For example, a microprocessor with a timer output set to a predetermined frequency, combined with analog circuitry to condition the signal before it is applied to the electrode could be used as the current generator. While the description herein relates to use of frequency as a parameter for implementing the concepts herein, depending on the waveform, other parameters such as duty cycle (for example, in square waveforms) can be relevant in determining the appropriate injection frequency. In this regard, when square wave waveforms are used, the time of the positive pulse width is known as the duty cycle of the period. Then, for a square wave waveform the positive or “ON” time is equal to the negative or “OFF” time so the duty cycle is 50% (half of its period). As frequency is equal to the reciprocal of the period, (1/T) the frequency of a square wave waveform can be characterized as follows:

$$Frequency = \frac{1}{ONtime + OFFtime} .$$

The therapy electrode 302 can be further configured to output a device-generated signal 312 as well as one or more treatment pulses 314 during, for example, execution of a treatment protocol when a patient is experiencing a cardiac event such as an arrhythmia.

As further shown in FIG. 3A, the therapy electrode 302 can be configured to receive one or more control signals 316 from a medical device controller as described herein. Based upon the control signals 316, the signal generation circuitry 308 can be configured to generate the device-generated signal 312 at a predetermined frequency as described herein. For example, the control signals 316 can include a clock signal or other similar output from a clock circuit that is configured to provide one or more frequencies to the signal generation circuitry 308 for use in generating the device-generated signal 312. Once generated, the device-generated signal 312 can be output to the patient via the skin contacting surface 304.

FIG. 3B illustrates a system 320 including a more detailed schematic view of a dedicated current injection electrode such as the current injection electrode 212 as shown in FIG. 2B and described above. For example, as shown in FIG. 3B, a current injection electrode 322 can include a skin contacting surface 324 and circuitry 326. As further described herein, the circuitry 326 can include signal generation circuitry 328 (for example, current generator circuitry configured to provide currents at one or more predetermined frequencies). For example, the current generator circuitry 328 can generate a sinusoidal wave, a square wave, a rectangular wave, a triangular wave, a sawtooth wave, a pulse wave, a chirp signal, or other such base as can be output via a function generator. For example, such a current generator can incorporate a precision waveform generator integrated circuit, that is configured to generate output waveforms such as sine, square and triangular output waveforms, with a minimum number of external components or adjustments. The circuit includes an operating frequency range that can be selected over eight decades of frequency, from 0.001 Hz to 300 kHz, by the correct choice of the external R-C components. A microprocessor with integrated timing peripherals can also be used. For example, a microprocessor with a timer output set to a predetermined frequency, combined with analog circuitry to condition the signal before it is applied to the electrode could be used as the current generator. While the description herein relates to use of frequency as a parameter for implementing the concepts herein, depending on the waveform, other parameters such as duty cycle (for example, in square waveforms) can be relevant in determining the appropriate injection frequency. In this regard, when square wave waveforms are used, the time of the positive pulse width is known as the duty cycle of the period. Then, for a square wave waveform the positive or “ON” time is equal to the negative or “OFF” time so the duty cycle is 50% (half of its period). As frequency is equal to the reciprocal of the period, (1/T) the frequency of a square wave waveform can be characterized as follows:

$$Frequency = \frac{1}{ONtime + OFFtime} .$$

As further shown in FIG. 3B, the current injection electrode 322 can be configured to receive one or more control signals 336 from a medical device controller as described herein. Based upon the control signals 336, the signal generation circuitry 328 can be configured to generate the device-generated signal 332 at a predetermined frequency as described herein. For example, the control signals 336 can include a clock signal or other similar output from a clock circuit that is configured to provide one or more frequencies to the signal generation circuitry 328 for use in generating the device-generated signal 332. Once generated, the device-generated signal 332 can be output to the patient via the skin contacting surface 324.

FIG. 4A illustrates system 400 including a more detailed schematic view of a sensing electrode such as one of sensing electrodes 204a to 204d as shown in FIG. 2A and described above. For example, as shown in FIG. 4A, an ECG assembly 402 can include a skin contacting substrate 404. In some examples, the skin contacting substrate 404 can be a dry substrate, for example, a substrate that is not attached to the patient's skin by an adhesive or where contact is mediated by conductive gel. In an example scenario, a dry substrate is placed directly on the skin and, as a result of the contact between the electrode and the skin, perspiration can accumulate on the electrode surface to provide an electrolytic connection with the patient. A dry substrate can be constructed from a housing configured to hold various circuit components and a treated, anodized metal surface configured to contact the patient's skin. For example, the treated, anodized metal surface can be treated with a tantalum pentoxide coating.

Depending on the design, a dry substrate can be configured to have a wide range of input impedances when in contact with a patient's skin. For example, the impedance as seen by the substrate when in contact with the patient's skin can be in excess of 400 ohms, typically in the range of tens to hundreds of megaohms. In certain implementations, the dry substrate can have an impedance range of 400 ohms to 10 megaohms. In some examples, a dry ECG substrate can be a high impedance electrode having an impedance range of 10 megaohms to 100 megaohms, 100 megaohms to 1.0 gigaohm, and 1.0 gigaohm to 10 gigaohms.

As described herein, the skin contacting substrate 404 can be configured to be in physical contact with the skin of the patient. Additionally, the ECG assembly 402 can include a circuitry 406. The circuitry 406 can including signal processing circuitry 408. In some examples, the signal processing circuitry 408 can be configured to receive signals from the skin contacting substrate 404 such as surface electrical signal 410 and process the received signals. The circuitry can also be configured to receive one or more control signals 412 from, for example, a medical device controller as described herein. In certain implementations, the circuitry 406 and signal processing circuitry 408 can be implemented as a printed circuit assembly manufactured in or otherwise printed on a dedicated circuit board.

FIG. 4B illustrates a system 420 including a more detailed schematic view of a sensing electrode such as one of sensing electrodes 204a to 204d as shown in FIG. 2B and described above. For example, as shown in FIG. 4B, an ECG assembly 422 can include a skin contacting substrate 424 and a signal transmission substrate 426. In some examples, the skin contacting substrate 424 can be a dry substrate, for example, a substrate that is not attached to the patient's skin by an adhesive or where contact is mediated by conductive gel.

As described herein, the skin contacting substrate 424 can be configured to be in physical contact with the skin of the patient. Similarly, the signal transmission substrate 426 can also be configured to be in physical contact with the skin of the patient. Additionally, the ECG assembly 422 can include a circuitry 428. The circuitry 428 can be configured to receive signals from the skin contacting substrate 424 (as well as, for example, control signals 438 from a medical device controller as described herein), process the received signals, and output one or more signals to the signal transmission substrate 426. In certain implementations, the circuitry 428 can be implemented as a printed circuit assembly manufactured in or otherwise printed on a dedicated circuit board.

As further shown in FIG. 4B, the circuitry 428 can include a signal processing circuitry 430. The signal processing circuitry 430 can be electrically coupled to the skin contacting substrate 424 and configured to receive one or more surface electrical signals 434 as sensed or otherwise received by the skin contacting substrate 424. Similarly, the circuitry 428 can include a signal generation circuitry 432. The signal generation circuitry 432 can be electrically coupled to the signal transmission substrate 426 and configured to provide a local version of the device-generated signal 436 for transmission into the patient's body. In certain implementations, the signal generation circuitry 432 can include one or more conditioning elements configured to condition the local biasing signal. For example, the conditioning elements can include one or more circuit elements such as resistors, capacitors, inductors, operational amplifiers, diodes, ground signals, voltage inputs, and other similar circuit elements.

As further shown in FIG. 4B, the signal transmission substrate 426 can be configured to output the local version of the device-generated signal 436 to the skin of the patient. The skin contacting substrate 424 can be configured to receive or otherwise sense the surface electrical signal 434. In some examples, the surface electrical signal 434 can include electrical signals generated by the body of the patient including, for example, signals indicative of cardiac activity of the patient as well as at least a portion of the local version of the device-generated signal 436 as output by the signal transmission substrate 426.

In certain implementations, a sensing electrode assembly can include a digital front end where additional signal conversion and processing can be performed at the sensing electrode assembly prior to the electrical signals being transmitted to, for example, a medical device controller for further processing. For example, FIG. 4C illustrates a system 440 including a detailed schematic view of a sensing electrode assembly that includes a digital front end. For example, as shown in FIG. 4C, an ECG assembly 442 can include a skin contacting substrate 444 and a signal transmission substrate 446. As described herein, the skin contacting substrate 444 can be configured to be in physical contact with the skin of the patient. Similarly, the signal transmission substrate 446 can also be configured to be in physical contact with the skin of the patient. Additionally, the ECG assembly 442 can include circuitry 448. The circuitry 448 can be configured to receive signals from the skin contacting substrate 444, process the received signals, and output one or more signals to the signal transmission substrate 446. In certain implementations, the circuitry 448 can be implemented as a printed circuit assembly manufactured in or otherwise printed on a dedicated circuit board.

As further shown in FIG. 4C, the circuitry 448 can include an signal processing circuitry 450. The signal processing circuitry 450 can be electrically coupled to the skin contacting substrate 444 and configured to receive one or more surface electrical signals 454 as sensed or otherwise received by the skin contacting substrate 444. Similarly, the circuitry 448 can include a signal generation circuitry 452. The signal generation circuitry 452 can be electrically coupled to the signal transmission substrate 446 and configured to provide a local device-generated signal 456 to the signal transmission substrate 446 for transmission into the patient's body. In certain implementations, the signal generation circuitry 452 can include one or more conditioning elements configured to condition the local device-generated signal 454. For example, the conditioning elements can include one or more circuit elements such as resistors, capacitors, inductors, operational amplifiers, diodes, ground signals, voltage inputs, and other similar circuit elements.

As further shown in FIG. 4C, the signal transmission substrate 446 is configured to output a local device-generated signal 456 to the skin of the patient. The skin contacting substrate 444 can be configured to receive or otherwise sense a surface electrical signal 454. In some examples, the surface electrical signal 454 includes electrical signals generated by the body of the patient including, for example, signals indicative of cardiac activity of the patient as well as at least a portion of the local device-generated signal 456 as output by the signal transmission substrate 446.

In some examples, as further shown in FIG. 4C, the output of the signal processing circuitry 450 can also be directed to an analog-to-digital converter (ADC) 458. The converter 458 can be configured to receive the output of the signal processing circuitry 450 and convert the signal to a digital signal. The digital output of the converter 458 can be output to a processor 460. The processor 460 can be configured to further process the digital signal to filter out various noise and other unwanted information. For example, the processor 460 can be configured to remove the device-generated signal 456 from the surface electrical signal 454 as described herein. The processor can then output one or more control signals 462 for further processing. In some examples, the output control signals 462 of the processor 460 can be a filtered pulse-width modulated signal for delivery to a medical device controller as described herein.

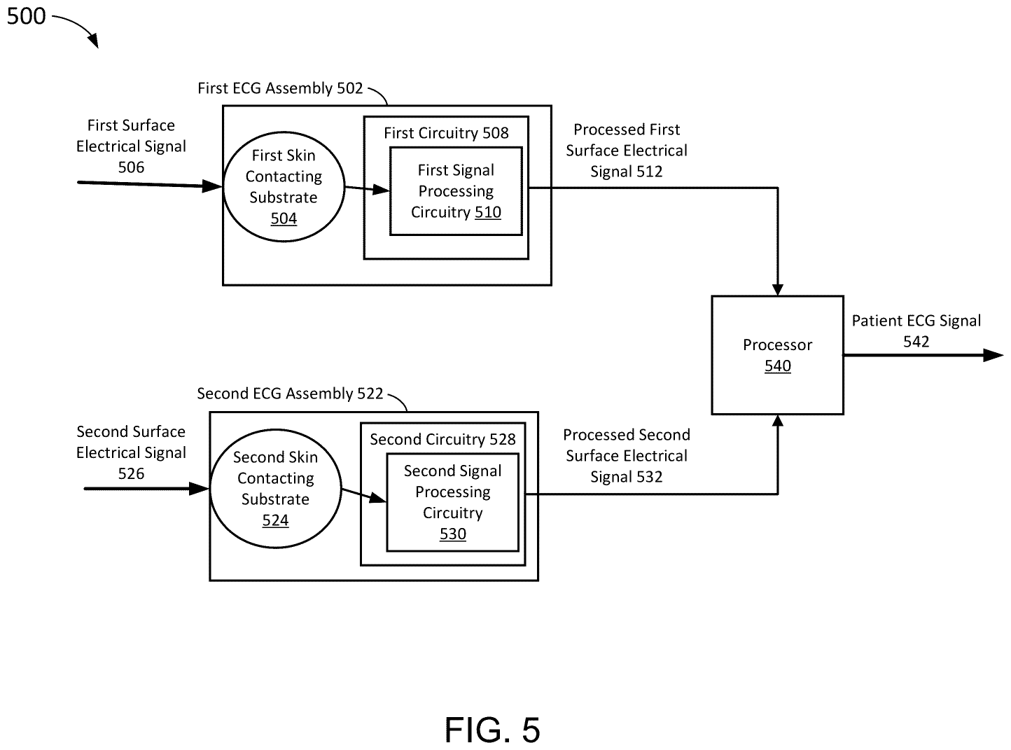

In certain examples, multiple sensor assemblies can be arranged into leads or sensor pairs, the outputs of which are used by a processor to determine one or more ECG metrics and an associated cardiac activity for a patient. For example, FIG. 5 illustrates a system 500 including two ECG assemblies as described herein. A first ECG assembly 502 can include a first skin contacting substrate 504 and first circuitry 508. The first skin contacting substrate 504 can be configured to receive a first surface electrical signal 506. The first circuitry 508 can include a first signal processing circuitry 510. The first circuitry 508 can be configured to process and analyze signals received from the first skin contacting substrate 504. Additionally, the first circuitry 508 can be configured to output a processed first surface electrical signal 512.

FIG. 5 further illustrates a second ECG assembly 522. The second ECG assembly 522 can include a second skin contacting substrate 524 and second circuitry 528. The second skin contacting substrate 524 can be configured to receive a second surface electrical signal 526. The second circuitry 528 can include a second signal processing circuitry 530. The second circuitry 528 can be configured to process and analyze signals received from the second skin contacting substrate 524. Additionally, the second circuitry 528 can be configured to output a processed second surface electrical signal 532.

As further shown in FIG. 5, a processor 540 can be configured to receive both the processed first surface electrical signal 512 and the processed second surface electrical signal 532. The processor 540 can be configured to further condition and process the received signals and output a patient ECG signal 542 for further analysis by, for example, a medical device controller as described herein. In certain implementations, the processor 540 can be integrated into a node such as belt node 202 as described herein above. In some examples, the processor 540 can be integrated into the medical device controller as described herein below. In other examples, an ECG assembly can be implemented as an active electrode that includes a local processor for conditioning the surface electrical signals directly at the sensing electrode. In such an example, the active electrode can be configured to output a signal similar to the patient ECG signal 542 for further processing by a medical device controller as described herein.

FIG. 6 illustrates an example of a medical device controller 600 that is configured to control components of the medical devices described herein as well as process signals received from, for example, one or more sensing electrodes as described herein. A brief introduction applicable to medical controllers in general will now be provided with reference to FIG. 6. A more detailed description of some of the components of the medical device controller 600 is provided for additional context in a sample medical device discussion further below.

More specifically, FIG. 6 illustrates an example component-level view of the medical device controller 600 included in, for example, a wearable medical device such as a WCD or an HWD as described herein. As shown in FIG. 6, the medical device controller 600 can include a housing 601. The housing 601 can house a therapy delivery circuitry 602 configured to provide one or more therapeutic shocks to a patient via at least two therapy electrodes 620 (for example, therapy electrode 106 and therapy electrodes 206a to 206c as described above), a data storage 604, a network interface 606, a user interface 608, and at least one rechargeable battery 610. The housing 601 can be further configured to house a sensor interface 612. The sensor interface 612, for example, interfaces with ECG sensing electrodes 622 (for example, sensing electrodes 104 and sensing electrodes 204a to 204d as described above). The sensor interface 612 also, for example, interfaces with non-ECG physiological sensors 623 such as vibrational sensors (for example, vibrational sensor 110), lung fluid sensors (for example, RF sensor 112), infrared and near-infrared-based pulse oximetry sensors, and blood pressure sensors, among others. The housing 601 can be further configured to house a cardiac event detector 616 and at least one processor 618.

In some examples, the patient monitoring medical device can include a medical device controller that includes like components as those described above but that does not include the therapy delivery circuitry 602 and the therapy electrodes 620 (shown in dotted lines). That is, in certain implementations, the medical device can include only ECG monitoring components and not provide therapy to the patient. In such implementations, for example, AMS, HFMS, CEM, EH, or MCT devices, the construction of the patient monitoring medical device is similar in many respects to the medical device controller 600 but need not include the therapy delivery circuitry 602 and associated therapy electrodes 620.

As described herein, when a sensing electrode senses a surface electrical signal for a patient, the signal includes both physiological data (such as the ECG signals) as well as the device-generated signal as described herein. The physiological data is processed to determine various information about the patient including, for example, cardiac activity. The device-generated signal can be further processed to determine, for example, whether one or more sensing electrodes have degraded contact with the patient's skin or have lost contact with the patient's skin altogether.

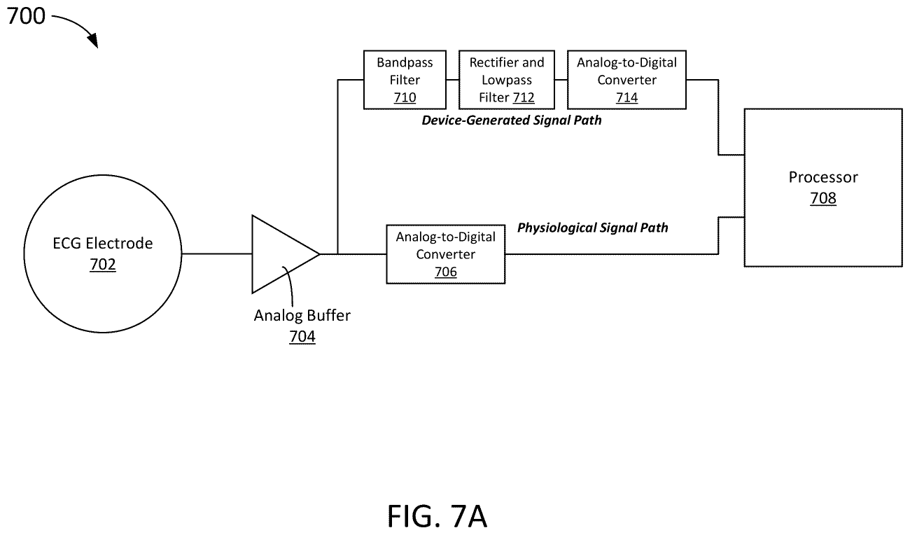

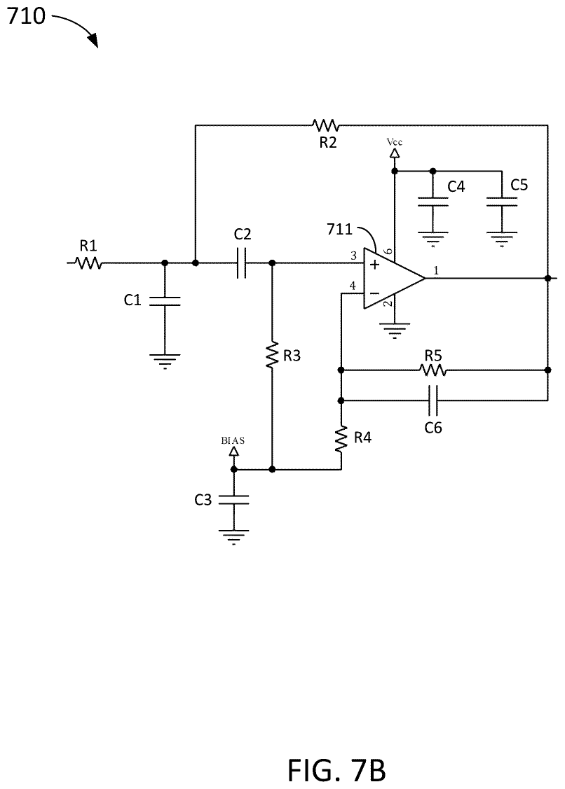

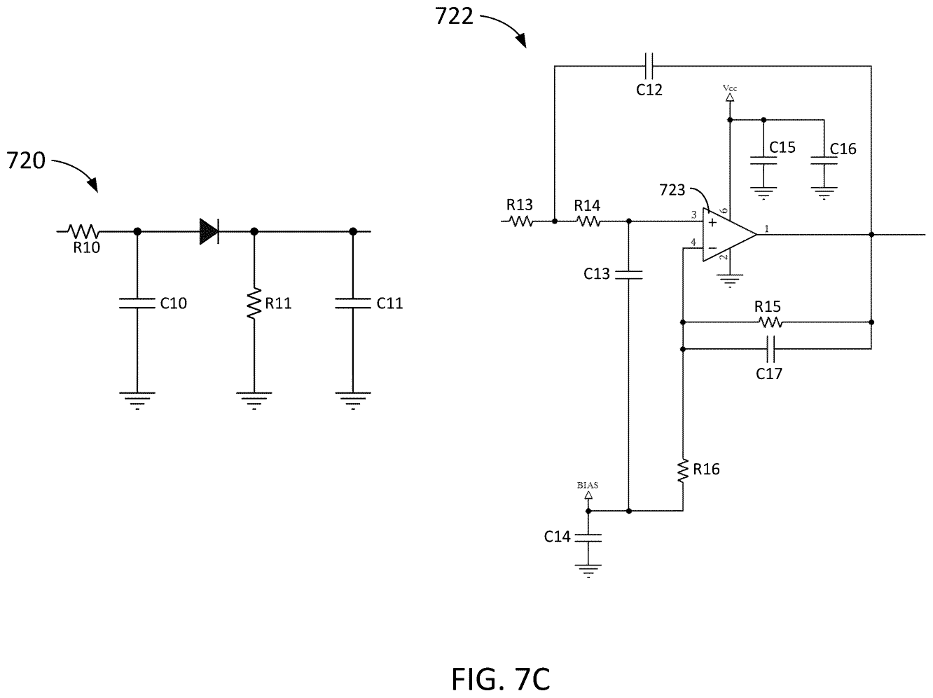

In order to properly process both the physiological signal and the device-generated signal, the surface electrical signal can be conditioned and divided into multiple processing paths. For example, FIG. 7A illustrates a sample system 700 that includes multiple processing paths for the surface electrical signals as described herein. As shown in FIG. 7A, surface electrical signals can be initially sensed by a sensing electrode such as ECG electrode 702. The ECG electrode 702 can be operably coupled to an analog buffer 704. The analog buffer 704 can be configured to receive the surface electrical signals and condition the signals for additionally processing. For example, the analog buffer 704 can be configured to divide the surface electrical signal into multiple copies of the signal, provide any conditioning such as signal amplification of the copies, and output the copies of the signals for further processing.

As further shown in FIG. 7A, the copies of the surface electrical signal can follow either a physiological signal path or a device-generated signal path. In the physiological signal path, the copy of the surface electrical signal can be passed to an ADC 706. In certain implementations, the ADC 706 can be configured to sample the surface electrical signal at a particular sampling rate. For example, to isolate the physiological signal data as described herein, the ADC 706 can be configured to sample the surface electrical signal at 2.0 kHz. However, as described herein, it should be noted that this sampling rate is provided by way of example only. The ADC 706 can then pass the sampled surface electrical signal to a processor 708 for further processing.

Additionally, a copy of the surface electrical signal can follow the device-generated signal path. As shown in FIG. 7A, the copy of the electrical signal can be passed to a bandpass filter 710. As described herein, the bandpass filter 710 can be configured to remove ambient noise from the surface electrical signal (for example, using a filter to remove ambient 60 Hz noise). Additionally, the bandpass filter 710 can be further configured to remove noise related to motion artifacts (for example, noise caused by movement of the ECG electrode 702 against the patient's skin) as well as the physiological signal from the surface electrical signal, thereby isolating the device-generated signal. The bandpass filter 710 can be operably coupled to a rectifier and lowpass filter circuit 712. The rectifier and lowpass filter circuit 712 can be configured to receive the isolated device-generated signal from the bandpass filter and to further process the signal. For example, the rectifier and lowpass filter circuit, in combination with an ADC 714, can be configured to condition and convert the isolated device-generated signal to a direct current signal. By converting to direct current, a processor such as processor 708 can sample the isolated device-generated signal at a lower sampling rate, thus saving power. For example, the processor 708 can sample the isolated device-generated signal at a dithered 10 Hz rate as dithering further helps to remove any noise that would otherwise show up in the signal.

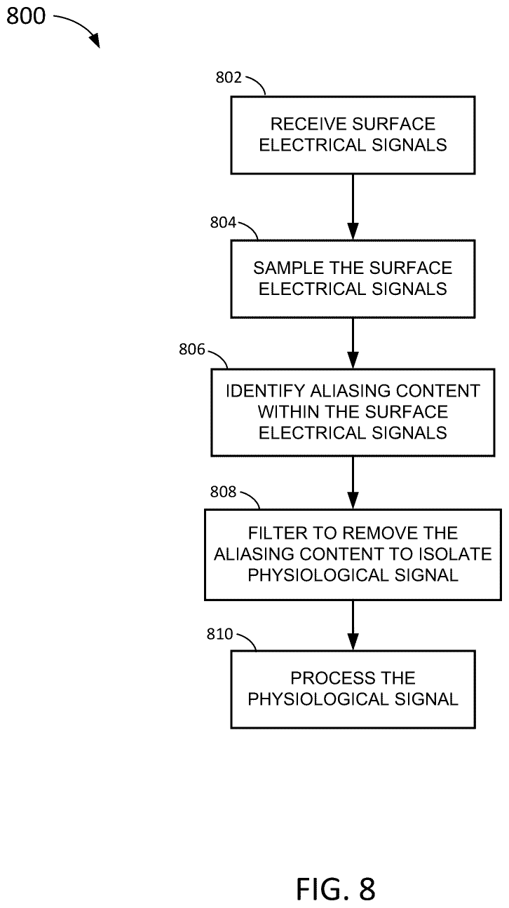

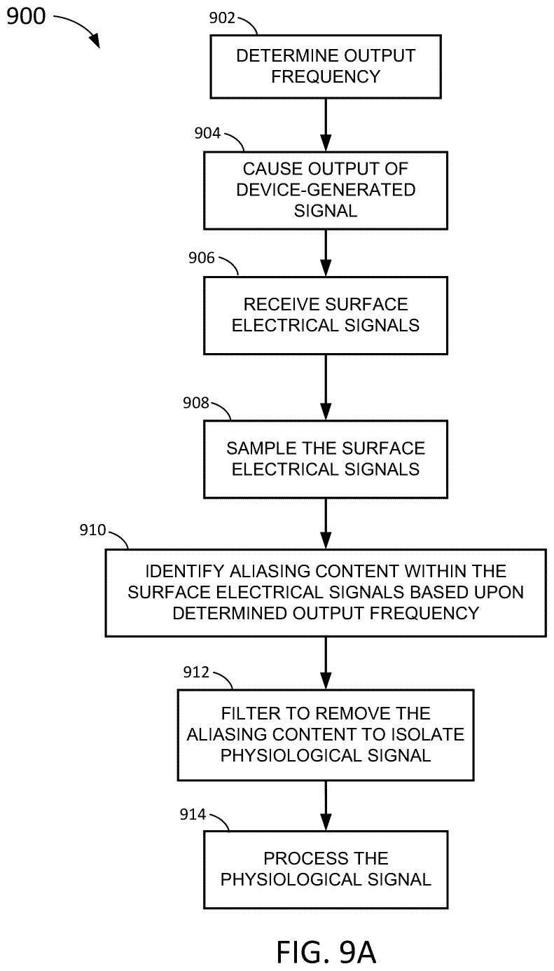

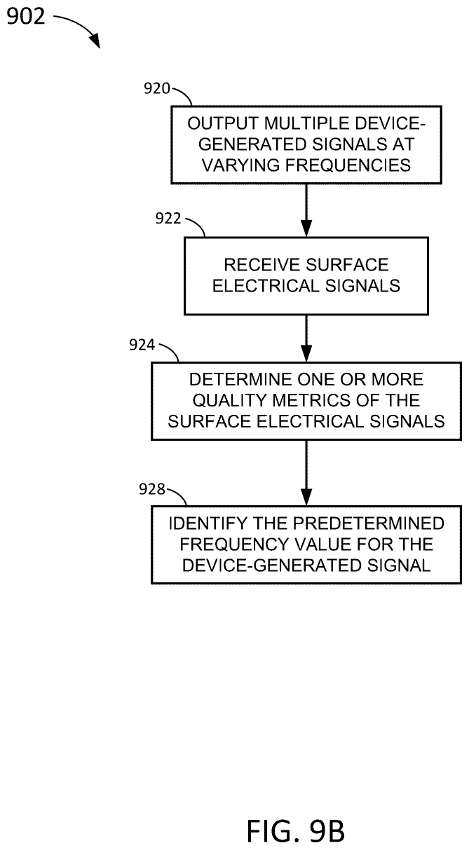

As further shown in FIG. 7A, the output of both the physiological signal path and the device-generated signal path are passed to processor 708. The processor 708 can be configured to receive the physiological signal and apply digital filtering to sample the physiological signal such that artifacts (for example, aliasing content) caused by the device-generated signal are removed and clean physiological data is isolated for further processing. Additional detail related to the additional sampling and processing of the surface electrical signals is provided in the discussions of, for examples, FIGS. 8, 9A, and 9B below.

As shown in FIG. 7A, the bandpass filter 710 can be configured such that it is configured to increase the amplitude of the device-generated signal frequency and attenuate other common noise frequencies such as 60 Hz noise. FIG. 7B illustrates a sample circuit diagram for the bandpass filter 710. It should be noted that the circuit diagram as shown in FIG. 7B is provided by way of example only and, in actual implementation, additional bandpass filter circuits can be used. Similarly, more than one bandpass filter circuit can be included in the bandpass filter 710, arranged in a cascading pattern to increase the overall effectiveness of the filter. As shown in FIG. 7B, one or more of the individual circuit components can be selected or otherwise tuned such that the output of the operational amplifier 711 of the bandpass filter 710 provides an appropriate and expected output. For example, the impedance values of the circuit resistors R1, R2, R3, R4, and R5 can be selected based upon testing and simulation to provide for both conditioned inputs to and an expected output from the operational amplifier 711. Similarly, the capacitive values of the circuit capacitors C1, C2, C3, C4, C5, and C6 can be selected based upon testing and simulation to provide for both conditioned inputs to and an expected output from the operational amplifier 711.

As further shown in FIG. 7A, the rectifier and lowpass filter circuit 712 can be configured to condition the signal for converting to direct current (the rectifier portion of the circuit) as well as to increase the signal output and reject any remaining alternate current components of the signal (the lowpass filter portion of the circuit). FIG. 7C illustrates sample circuits 720 and 722 including one example circuit layout for the rectifier and the lowpass filter respectively. As shown in circuit 720, a simple rectifier circuit can be used. However, it should be noted that, in some example, a switching or synchronous rectifier can be used. Additionally, it should be noted that the individual circuit components included in the rectifier circuit 720 (for example, R10, R11, C10, and C11) can be selected based upon testing and simulation of the rectifier circuit such that the output of the rectifier is properly conditioned for further processing.

As further shown in FIG. 7C, the lowpass filter circuit 722 can be configured such that it is configured to increase the output of the rectifier circuit 720 and reject any remaining alternate current signal components. It should be noted that the circuit diagram 722 as shown in FIG. 7C is provided by way of example only and, in actual implementation, additional lowpass filter circuits can be used. Similarly, more than one lowpass filter circuit can be included in the lowpass circuit 722, arranged in a predetermined cascading pattern to increase an overall effectiveness of the filter. For example, more than one of the circuit 722 shown in FIG. 7C can be placed such that two or more stages of circuit 722 are connected together in a way that output of a previous stage is connected to input of a next stage and so on. As shown in FIG. 7C, one or more of the individual circuit components can be selected or otherwise tuned such that the output of the operational amplifier 723 of the lowpass filter circuit 722 provides an appropriate and expected output. For example, the impedance values of the circuit resistors R13, R14, R15, and R16 can be selected based upon testing and simulation to provide for both conditioned inputs to and an expected output from the operational amplifier 723. Similarly, the capacitive values of the circuit capacitors C12, C13, C14, C15, C16, and C17 can be selected based upon testing and simulation to provide for both conditioned inputs to and an expected output from the operational amplifier 723.

FIG. 8 illustrates a sample process 800 that can be used by a processor, such as a processor like the processor 618 as shown in FIG. 6 and discussed above, to analyze surface electrical signals as sensed by, for example, a sensing electrode as described herein. The processor can be further configured to sample the surface electrical signals such that aliasing content caused by the device-generated signal is identified and filtered from the surface electrical signals, thereby providing processed and conditioned physiological signals for further processing to determine, for example, cardiac activity of the patient.

As shown in FIG. 8, the processor can receive 802 surface electrical signals from, for example, a sensing electrode as described herein. The processor can be further configured to sample 804 the surface electrical signals at a predetermined sampling rate. As described herein, the sampling rate can be selected based upon the frequency of the device-generated signal as injected into the patient's body. In certain implementations, the sampling rate of the surface electrical signal can be selected such that the frequency of the device-generated signal is an integer multiple of the sampling rate. In certain implementations, the sampling rate can be selected such that the device-generated signal has a predetermined frequency that is an integer multiple of the sampling rate including, for example, an integer multiple of two, three, four, five, or six. More specifically, if the sampling rate is selected as 2.0 kHz, the predetermined frequency of the device-generated signal can be set as 8.0 kHz (for example, having an integer multiple of four).

As further shown in FIG. 8, based upon the sampling of the surface electrical signals, the processor can identify 806 aliasing content within the surface electrical signals as caused by the device-generated signal injected into the patient's body. Once identified, the processor can filter 808 the surface electrical signals to remove the aliasing content. In some examples, the processor can be configured to continuously isolate the device-generated signal from the surface electrical signals. In another example, the processor can be configured to periodically isolate the device-generated signal from the surface electrical signals. For example, the processor can be configured to isolate the device-generated signal every 50 ms, every 100 ms, every 250 ms, every 500 ms, or every 1000 ms.

Based upon the filtering, the processor can then isolate a physiological signal for the patient. This physiological signal may be indicative of, for example, the current cardiac activity of the patient. The processor can further process 810 the physiological signal to determine, for example, if the patient is experiencing a cardiac event such as an arrhythmia. Additionally, the processor can further process the aliasing content as isolated from the surface electric signals to determine, for example, if one or more of the sensing electrodes have fallen off of the patient or otherwise have compromised contact with the patient's skin.

FIG. 9A illustrates a sample process 900 that can be used by a processor, such processor 618 as shown in FIG. 6 and discussed above, to analyze surface electrical signals. More specifically, the process 900 illustrated in FIG. 9A is a detailed process flow similar to process 800 as shown in FIG. 8 and described above. As shown in FIG. 9A, the processor can be further configured to determine and output frequency for the device-generated signal and to cause the output of the device-generated signal by one or more electrodes as described herein. By determining the output frequency of the device-generated signal, the processor can further determine the rate of sampling of the sensed electrical signals as described herein such that any aliasing content caused by the device-generated signal is quickly and efficiently removed from the surface electrical signals.

As shown in FIG. 9A, the processor can determine 902 the output frequency of the device-generated signal as described herein. For example, the predetermined frequency of the device-generated signal can be an integer multiple of the predetermined sampling rate used to further process any sensed surface electrical signals. As such, the processor can be configured to determine 902 the output frequency of the device-generated signal based upon the sampling rate to be used to further process the surface electrical signals. For example, if the processor is configured to sample the surface electrical signals at a sampling rate of 2.0 kHz, the processor can be configured to determine 902 an output frequency for the device-generated signal that is an integer multiple of 2.0 kHz. In some examples, the processor can perform some or all of the signal sampling in software. In such an example, the sampling rate can be dynamically adjusted by the processor. In such an example, the predetermined frequency of the device-generated signal can be adjusted as well, such that the device-generated signal has a frequency that is an integer multiple of the sampling rate. As such, and as described herein, by providing a device-generated signal at an integer multiple of the signal sampling rate, aliasing content (and the associated signal noise) can be filtered from sensed surface electrical signals when monitoring patient cardiac activity.

As further shown in FIG. 9A, the processor can cause 904 output of the device-generated signal by one or more electrodes as described herein. For example, as shown in FIG. 2A, the processor can cause 904 one or more therapy electrodes to output the device-generated signal at the predetermined frequency. In other examples, such as the example shown in FIG. 2B, the processor can cause 904 one or more sensing electrodes to output the device-generated signal at the predetermined frequency. The processor can receive 906 surface electrical signals from, for example, a sensing electrode as described herein. The processor can be further configured to sample 908 the surface electrical signals at a predetermined sampling rate. As described herein, the sampling rate of the surface electrical signal can be selected such that the frequency of the device-generated signal is an integer multiple of the sampling rate. In certain implementations, the sampling rate can be selected such that the device-generated signal has a predetermined frequency that is an integer multiple of the sampling rate including, for example, an integer multiple of two, three, four, five, or six. More specifically, if the sampling rate is selected as 2.0 kHz, the predetermined frequency of the device-generated signal can be set as 8.0 kHz (for example, having an integer multiple of four).

In some examples as described herein, the processor and the signal generation circuitry can be configured to be running asynchronously. For example, the device-generated signal as generated by the signal generation circuitry can be in any arbitrary phase relative to the device-generated signal as detected within the at least one surface electrical signal (for example, the detection circuitry is not provided with and does not operate on phase information of the original device-generated signal). In such an example, while the processor is configured with the frequency of the device-generated signal, the processor can still efficiently identify and isolate the aliasing content within the surface electrical signal as generated by the device-generated signal as described herein. The processor does not need information regarding the phase of the device-generated signal and is therefore indifferent to (for example, does not process) the phase information. In this regard, the signal generation circuitry, for example, current generator, can inject a signal at a predetermined frequency. The detection circuitry at one or more of the therapy or sensing electrodes is able to pick up the injected signal regardless of the phase of the device-generated signal. In some examples, the detection circuitry is not synchronized to the signal generation circuitry, for example, the current generator, and as such is indifferent with respect to the original phase of the device-generated signal. In this manner, an unsynchronized detection circuit is able to effectively pick up fall off signals and filter aliasing content based on the frequency content of the fall off signal.

In other examples, the processor and the signal generation circuitry can be configured to run synchronously. In such an example, a clock circuit can be provided to both the processor and the signal generation circuitry. The processor and the signal generation circuitry can be configured to operate in concert (as defined by the clock circuit) such that the device-generated signal as generated by the signal generation circuitry is in the same phase as the device-generated signal as detected (and later sampled as described herein) within the surface electrical signal. In such an example, the processor can quickly and efficiently identify and isolate the aliasing content within the surface electrical signal by aligning the phase of the signal with the device-generated signal and filtering for the aliasing content as described herein.

As further shown in FIG. 9A, based upon the sampling of the surface electrical signals, the processor can identify 910 aliasing content within the surface electrical signals as caused by the device-generated signal injected into the patient's body. Once identified, the processor can filter 912 the surface electrical signals to remove the aliasing content. In some examples, the processor can be configured to continuously isolate the device-generated signal from the surface electrical signals. In other examples, the processor can be configured to periodically isolate the device-generated signal from the surface electrical signals. For example, the processor can be configured to isolate the device-generated signal every 50 ms, every 100 ms, every 250 ms, every 500 ms, or every 1000 ms.

Based upon the filtering, the processor can then isolate the physiological signal for the patient, the physiological signal indicative of, for example, the current cardiac activity of the patient. The processor can further process 914 the physiological signal to determine, for example, if the patient is experiencing a cardiac event such as an arrhythmia. Additionally, the processor can further process the aliasing content as isolated from the surface electric signals to determine, for example, if one or more of the sensing electrodes have fallen off of the patient or otherwise have compromised contact with the patient's skin.

In some examples as described herein, the processor can be configured to dynamically determine the frequency of the device-generated signal. For example, as shown in FIG. 9B, a specific process can be provided for determining 902 the frequency of the device-generated signal. For example, the processor can be configured to dynamically determine the frequency of the device-generated signal based upon current operating characteristics of the medical device and/or one or more quality metrics as measured by the processor.

For example, as shown in FIG. 9B, the processor can be configured to output 920 multiple device-generated signals at varying frequencies selected from a range of predetermined frequency values. For example, the processor can output 920 the device-generated signal for a period of time at each frequency within the range of predetermined frequency values. In certain implementations, the processor can output each of the frequency values for, for example, 1 second, 5 seconds, 10 seconds, 15 seconds, seconds, 25 seconds, or 30 seconds. In certain examples, the range of predetermined frequency values can include frequencies such as 100 Hz, 200 Hz, 300 Hz, 400 Hz, 500 Hz, 1.0 kHz, 2.0 kHz, 4.0 kHz, and 8.0 kHz.

For each of the output frequency values, the processor can receive 922 one or more corresponding surface electrical signals. For each of the received surface electrical signals, the processor can determine 924 one or more quality metrics of the surface electrical signal. For example, the processor can determine a signal to noise ratio for the received surface electrical signals, an ECG recognition metric for the received surface electrical signals, and other similar quality metrics. Each of the quality metrics can have an associated threshold value. As such, the processor can be configured to eliminate the frequency associated with the device-generated signal that caused the quality metric to transgress the threshold. For example, the processor can store a signal-to-noise ratio threshold of 0.95 (on a scale of 0.0 to 1.0 where 0.0 is all noise and 1.0 is a noiseless signal). If the processor determines that an output frequency has a signal-to-noise ratio below 0.95, the processor can eliminate that output frequency and test another frequency. In such an example, the processor can iteratively step through each of the range of frequency values to identify 928 the predetermined frequency value for the device-generated signal that provides the best overall surface electrical signal for further processing as described herein.

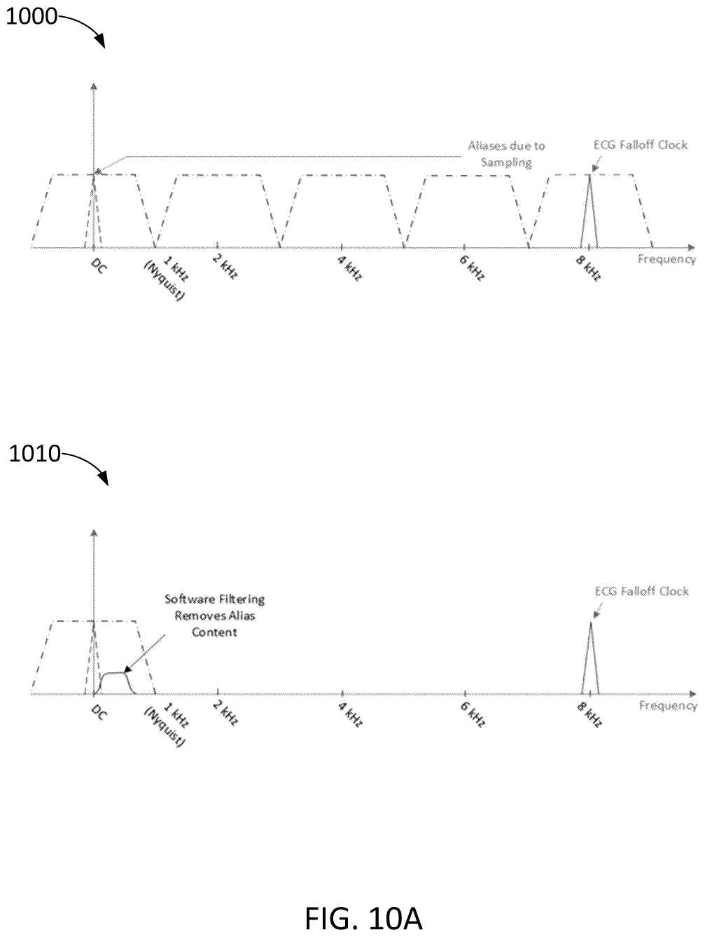

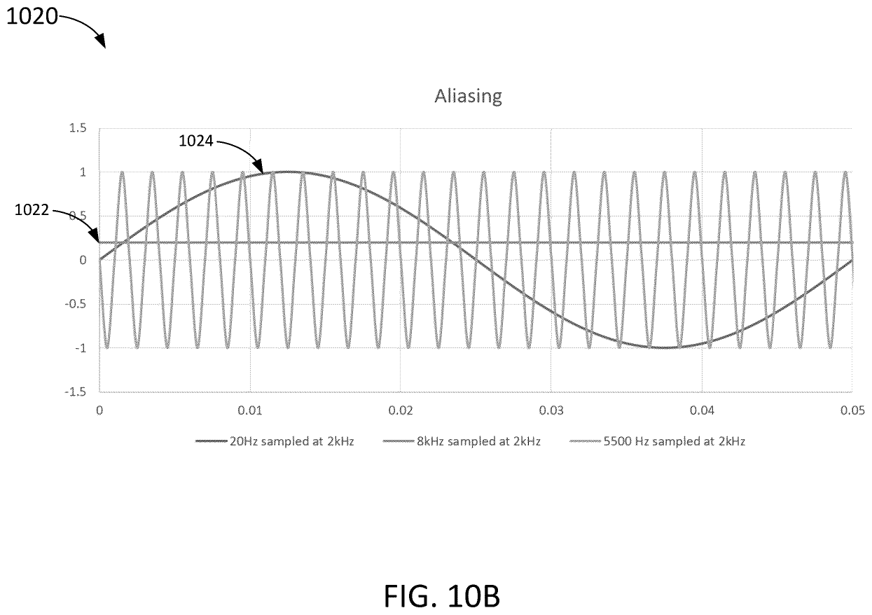

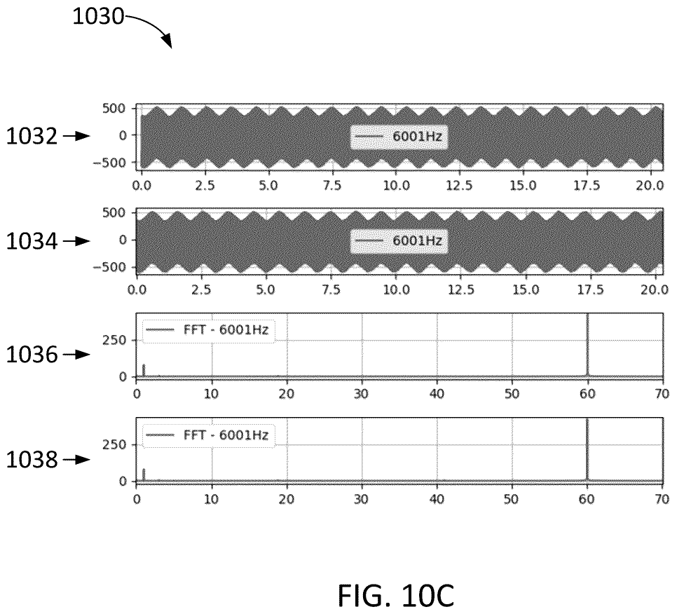

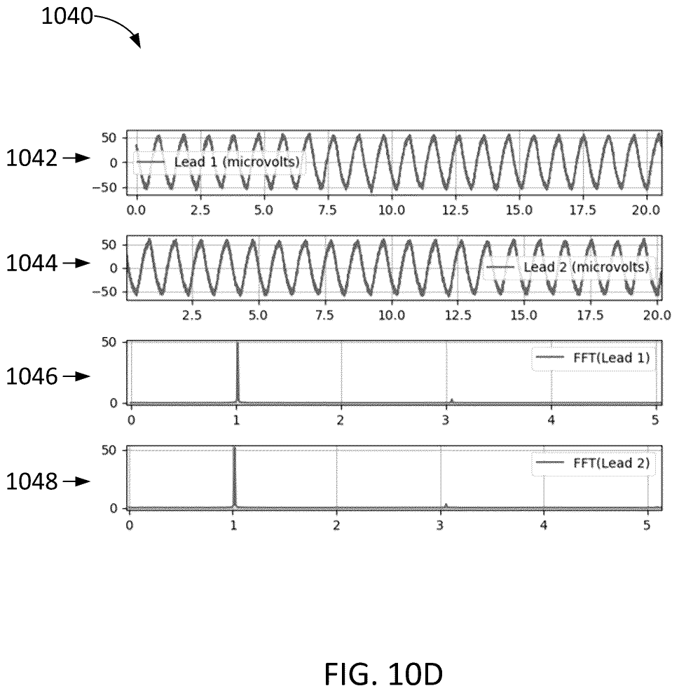

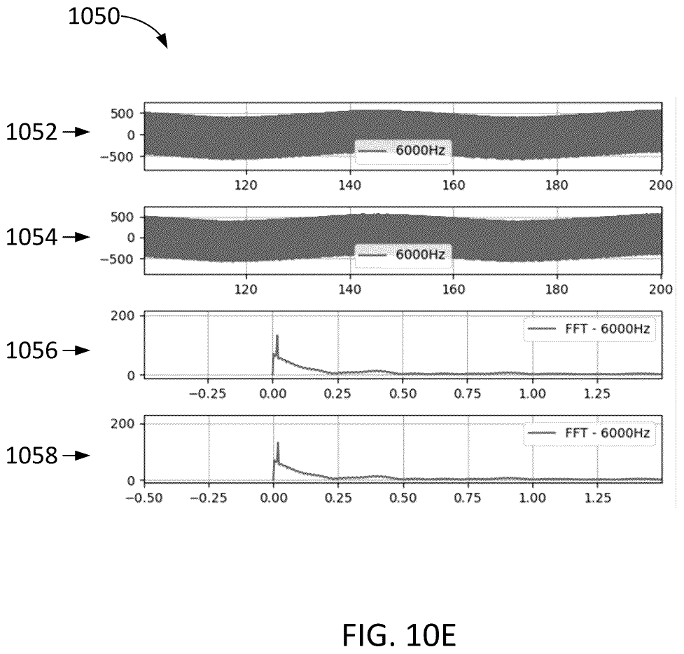

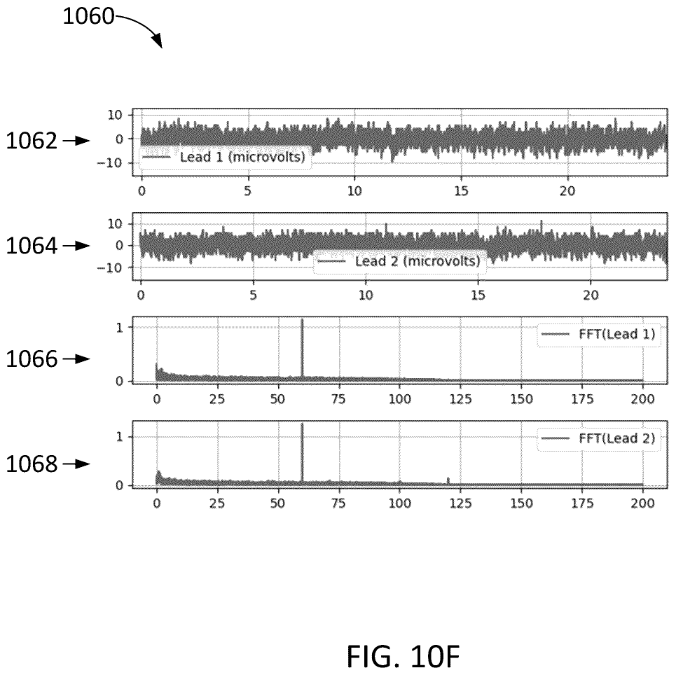

FIG. 10A illustrates sample output graphs 1000 and 1010 showing example sensed electrical surface signals both prior to and after isolating and removing the aliasing content as caused by the device-generated signal as described herein. For example, as shown in graph 1000, an 8.0 kHz device-generated signal has been injected into the patient's body (shown in graph 1000 as the ECG falloff clock signal). Various aliasing content as caused by the device-generated signal is also included in the surface electrical signal (shown by the dotted square peaks centered around 2.0 kHz, 4.0 kHz, 6.0 kHz, and 8.0 kHz). By using a sampling rate of 2.0 kHz, the processor can use software filtering to remove the alias content as caused by the device-generated signal. For example, as shown in graph 1010, the aliasing content centered around 2.0 kHz, 4.0 kHz, 6.0 kHz, and 8.0 kHz has been removed and the patient physiological data (starting around 0.0 kHz and ending around 600 Hz) has been identified and isolated for further processing by, for example, a medical device controller as described herein.

FIG. 10B further illustrates sample output graph 1020. In this example, the physiological signal data is sampled at 2.0 kHz and the device-generated signal is output at 8.0 kHz (for example, the sampling rate times the integer four). As shown in Figure when the combined surface electrical signal (including the physiological signal and the device-generated signal as described herein) is sampled at 2.0 kHz, the 8.0 kHz device-generated signal aliases to a direct current signal represented by line 1022. In such an example, the direct current alias content can be removed using, for example, a high-pass filter in software without impacting the physiological signal.

Additionally, in some examples, the device-generated signal can be offset a particular amount. For example, as above, the physiological data can be sampled at 2.0 kHz and the device-generated signal can be sampled at 5.5 kHz (which is the sampling rate times the integer three and then subtracting 500 Hz). In such an example, when the surface electrical signal is sampled at 2.0 kHz, the 5.5 kHz device-generated signal is aliased to 500 Hz (as shown by wave 1024 in FIG. 10B). As this frequency is much higher than needed for identifying the physiological signal, the 500 Hz aliasing content can be removed using, for example, a lowpass filter in software without impacting the physiological signal. To summarize the above example, the ECG data is sampled at F1=2 kHz, and the device-generated fall off signal source is set to

(F1×n)−500 Hz=5500 Hz,

where n=3.

When the ECG and device-generated fall off signal is sampled at 2 kHz, the 5500 Hz device-generated fall off signal is aliased to 500 Hz, which is higher than needed for ECG data. This 500 Hz alias can be removed in software with a lowpass filter without impacting the ECG signal.

FIG. 10C is a set of graphs 1030 depicting raw data acquired by electrodes in an experimental ECG acquisition system where the device-generated signal is not synchronized with the surface electrical signal sampling rate. In this example, the device-generated signal comprises a 6001 Hz square wave, and the raw electrode data sample rate is 2000 Hz. Despite anti-aliasing filters, some of the attenuated device-generated signal content still gets sampled by the electrode analog-to-digital converter. In this case, the electrode anti-aliasing filter removes the sharp edges so that the acquired waveforms 1032 and 1034 appear sinusoidal. In examples, the aliased 1 Hz content can be combined with ambient 60 Hz (as there is no 60 Hz filtering in this example). Both 1 Hz alias and environmental 60 Hz content are seen in the FFT graphs 1036 and 1038.

FIG. 10D is a set of graphs 1040 depicting the raw data illustrated in FIG. 10C once the data is formed into ECG leads (“Lead 1” and “Lead 2”). As shown, the experimental waveforms 1042 and 1044 include the 1 Hz alias content (as illustrated in FFT graphs 1046 and 1048), which can be challenging to filter out and still meet predetermined safety standards for ECG (for example, a passband of 0.67 Hz to 40 Hz is commonly referenced in 60601-series standards, such as IEC 60601-2-47:2012, Edition 2.0, “Medical electrical equipment—Part 2-47: Particular requirements for the basic safety and essential performance of ambulatory electrocardiographic systems”, subclause 201.12.4.4.108, “Frequency Response”). In some examples, ECG systems as described herein can use periodic (for example, not continuous) device-generated signals to detect lead-off or fall off events. In such systems, the 1 Hz alias may not have as much impact on operation and performance of the system. In other examples, however, ECG systems as described herein can be implemented for continuous monitoring of fall off or lead off. In such systems, it is desirable to avoid the 1 Hz alias content otherwise the system noise floor can quickly increase to unacceptable levels.

FIG. 10E is a set of graphs 1050 depicting raw data acquired by electrodes in an experimental ECG acquisition system where the device-generated signal is nearly synchronized with the surface electrical signal sampling rate. As shown in FIG. 10E, the raw electrode data shown in waveforms 1052 and 1054 includes a 0.02 Hz alias from the nearly synchronized device-generated signal. In this example, the device-generated signal frequency is 6000 Hz and the electrode sample rate remains at 2000 Hz (an integer multiple of 3). The alias in this scenario approaches direct current (DC) frequency as shown. In this example, it is noted that the actual alias content in this example is 0.02 Hz (as illustrated in FFT graphs 1056 and 1058) because the 6000 Hz device-generated signal rate uses a different clock than the electrode's 2000 Hz analog-to-digital converter sample rate. If they were derived from the same clock, the resulting alias would be at 0 Hz (that is, DC frequency).

FIG. 10F is a set of graphs 1060 depicting the raw data illustrated in FIG. 10E once the data is formed into ECG leads (“Lead 1” and “Lead 2”). In this example, the experimental waveforms 1062 and 1064 include no discernible alias (as illustrated in FFT graphs 1066 and 1068). Once the leads are formed, a 0.67 Hz digital high-pass filter has been implemented to remove the 0.02 Hz alias (as it would a 0 Hz alias) so that nothing remains in the acquired signal. It is noted that this data also includes 60 Hz environmental content as this example does not include a 60 Hz filter.

The teachings of the present disclosure can be generally applied to external medical monitoring and/or treatment devices that include one or more sensors as described herein. Such external medical devices can include, for example, ambulatory medical devices as described herein that are capable of and designed for moving with the patient as the patient goes about his or her daily routine. An example ambulatory medical device can be a wearable medical device such as a WCD, a wearable cardiac monitoring device, an in-hospital device such as an HWD, a short-term wearable cardiac monitoring and/or therapeutic device, mobile cardiac event monitoring devices, and other similar wearable medical devices.

The wearable medical device can be capable of continuous use by the patient. In some implementations, the continuous use can be substantially or nearly continuous in nature. That is, the wearable medical device can be continuously used, except for sporadic periods during which the use temporarily ceases (for example, while the patient bathes, while the patient is refit with a new and/or a different garment, while the battery is charged/changed, while the garment is laundered, and so forth). Such substantially or nearly continuous use as described herein may nonetheless be considered continuous use. For example, the wearable medical device can be configured to be worn by a patient for as many as 24 hours a day. In some implementations, the patient can remove the wearable medical device for a short portion of the day (for example, for half an hour to bathe). In such an example, “nearly continuous” can include 23.5 hours a day of wear with a half hour removal period.

Further, the wearable medical device can be configured as a long term or extended use medical device. Such devices can be configured to be used by the patient for an extended period of several days, weeks, months, or even years. In some examples, the wearable medical device can be used by a patient for an extended period of at least one week. In some examples, the wearable medical device can be used by a patient for an extended period of at least 30 days. In some examples, the wearable medical device can be used by a patient for an extended period of at least one month. In some examples, the wearable medical device can be used by a patient for an extended period of at least two months. In some examples, the wearable medical device can be used by a patient for an extended period of at least three months. In some examples, the wearable medical device can be used by a patient for an extended period of at least six months. In some examples, the wearable medical device can be used by a patient for an extended period of at least one year. In some implementations, the extended use can be uninterrupted until a physician or other healthcare provider (HCP) provides specific instruction to the patient to stop use of the wearable medical device.

Regardless of the extended period of wear, the use of the wearable medical device can include continuous or nearly continuous wear by the patient as described above. For example, the continuous use can include continuous wear or attachment of the wearable medical device to the patient, for example, through one or more of the electrodes as described herein, during both periods of monitoring and periods when the device may not be monitoring the patient but is otherwise still worn by or otherwise attached to the patient. The wearable medical device can be configured to continuously monitor the patient for cardiac-related information (for example, ECG information, including arrhythmia information, cardio-vibrations, and so forth) and/or non-cardiac information (for example, blood oxygen, the patient's temperature, glucose levels, tissue fluid levels, and/or lung vibrations). The wearable medical device can carry out its monitoring in periodic or aperiodic time intervals or times. For example, the monitoring during intervals or times can be triggered by a user action or another event.

As noted above, the wearable medical device can be configured to monitor other non-ECG physiologic parameters of the patient in addition to cardiac related parameters. For example, the wearable medical device can be configured to monitor, for example, pulmonary-vibrations (for example, using microphones and/or accelerometers), breath vibrations, sleep related parameters (for example, snoring, sleep apnea), tissue fluids (for example, using radio-frequency transmitters and sensors), among others.

Other example wearable medical devices include automated cardiac monitors and/or defibrillators for use in certain specialized conditions and/or environments such as in combat zones or within emergency vehicles. Such devices can be configured so that they can be used immediately (or substantially immediately) in a life-saving emergency. In some examples, the ambulatory medical devices described herein can be pacing-enabled, for example, capable of providing therapeutic pacing pulses to the patient. In some examples, the ambulatory medical devices can be configured to monitor for and/or measure ECG metrics including, for example, heart rate (such as average, median, mode, or other statistical measure of the heart rate, and/or maximum, minimum, resting, pre-exercise, and post-exercise heart rate values and/or ranges), heart rate variability metrics, premature ventricular contraction (PVC) burden or counts, atrial fibrillation burden metrics, pauses, heart rate turbulence, QRS height, QRS width, changes in a size or shape of morphology of the ECG information, cosine R-T, artificial pacing, QT interval, QT variability, T wave width, T wave alternans, T-wave variability, and ST segment changes.

As noted above, FIG. 6 illustrates an example component-level view of a medical device controller 600 included in, for example, a wearable medical device. As further shown in FIG. 6, the therapy delivery circuitry 602 can be coupled to one or more electrodes 620 configured to provide therapy to the patient. For example, the therapy delivery circuitry 602 can include, or be operably connected to, circuitry components that are configured to generate and provide an electrical therapeutic shock. The circuitry components can include, for example, resistors, capacitors, relays and/or switches, electrical bridges such as an h-bridge (for example, including a plurality of insulated gate bipolar transistors or IGBTs), voltage and/or current measuring components, and other similar circuitry components arranged and connected such that the circuitry components work in concert with the therapy delivery circuitry and under control of one or more processors (for example, processor 618) to provide, for example, at least one therapeutic shock to the patient including one or more pacing, cardioversion, or defibrillation therapeutic pulses.

Pacing pulses can be used to treat cardiac arrhythmia conditions such as bradycardia (for example, less than 30 beats per minute) and tachycardia (for example, more than 150 beats per minute) using, for example, fixed rate pacing, demand pacing, anti-tachycardia pacing, and the like. Defibrillation pulses can be used to treat ventricular tachycardia and/or ventricular fibrillation.

The capacitors can include a parallel-connected capacitor bank consisting of a plurality of capacitors (for example, two, three, four or more capacitors). In some examples, the capacitors can include a single film or electrolytic capacitor as a series connected device including a bank of the same capacitors. These capacitors can be switched into a series connection during discharge for a defibrillation pulse. For example, a single capacitor of approximately 140 pF or larger, or four capacitors of approximately 650 pF can be used. The capacitors can have a 1600 VDC or higher rating for a single capacitor, or a surge rating between approximately 350 VDC to 500 VDC for paralleled capacitors and can be charged in approximately 15 seconds to 30 seconds from a battery pack.

For example, each defibrillation pulse can deliver between 60 joules to 180 joules of energy. In some implementations, the defibrillating pulse can be a biphasic truncated exponential waveform, whereby the signal can switch between a positive and a negative portion (for example, charge directions). This type of waveform can be effective at defibrillating patients at lower energy levels when compared to other types of defibrillation pulses (for example, such as monophasic pulses). For example, an amplitude and a width of the two phases of the energy waveform can be automatically adjusted to deliver a precise energy amount (for example, 150 joules) regardless of the patient's body impedance. The therapy delivery circuitry 602 can be configured to perform the switching and pulse delivery operations, for example, under control of the processor 618. As the energy is delivered to the patient, the amount of energy being delivered can be tracked. For example, the amount of energy can be kept to a predetermined constant value even as the pulse waveform is dynamically controlled based on factors such as the patient's body impedance while the pulse is being delivered.

In certain examples, the therapy delivery circuitry 602 can be configured to deliver a set of cardioversion pulses to correct, for example, an improperly beating heart. When compared to defibrillation as described above, cardioversion typically includes a less powerful shock that is delivered at a certain frequency to mimic a heart's normal rhythm.

The data storage 604 can include one or more of non-transitory computer-readable media, such as flash memory, solid state memory, magnetic memory, optical memory, cache memory, combinations thereof, and others. The data storage 604 can be configured to store executable instructions and data used for operation of the medical device controller 600. In certain examples, the data storage can include executable instructions that, when executed, are configured to cause the processor 618 to perform one or more operations. In some examples, the data storage 604 can be configured to store information such as ECG data as received from, for example, the sensing electrode interface.

In some examples, the network interface 606 can facilitate the communication of information between the medical device controller 600 and one or more other devices or entities over a communications network. For example, where the medical device controller 600 is included in an ambulatory medical device, the network interface 606 can be configured to communicate with a remote computing device such as a remote server or other similar computing device. The network interface 606 can include communications circuitry for transmitting data in accordance with a Bluetooth® wireless standard for exchanging such data over short distances to an intermediary device. For example, such an intermediary device can be configured as a base station, a “hotspot” device, a smartphone, a tablet, a portable computing device, and/or other devices in proximity of the wearable medical device including the medical device controller 600. The intermediary device(s) may, in turn, communicate the data to a remote server over a broadband cellular network communications link. The communications link may implement broadband cellular technology (for example, 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with a remote server over a Wi-Fi® communications link based on the IEEE 802.11 standard.

In certain examples, the user interface 608 can include one or more physical interface devices such as input devices, output devices, and combination input/output devices and a software stack configured to drive operation of the devices. These user interface elements can render visual, audio, and/or tactile content. Thus, the user interface 608 can receive input or provide output, thereby enabling a user to interact with the medical device controller 600.

The medical device controller 600 can also include at least one rechargeable battery 610 configured to provide power to one or more components integrated in the medical device controller 600. The rechargeable battery 610 can include a rechargeable multi-cell battery pack. In one example implementation, the rechargeable battery 610 can include three or more 2200 mAh lithium ion cells that provide electrical power to the other device components within the medical device controller 600. For example, the rechargeable battery 610 can provide its power output in a range of between 20 mA to 1000 mA (for example, 40 mA) output and can support 24 hours, 48 hours, 72 hours, or more, of runtime between charges. In certain implementations, the battery capacity, runtime, and type (for example, lithium ion, nickel-cadmium, or nickel-metal hydride) can be changed to best fit the specific application of the medical device controller 600.

The sensor interface 612 can include physiological signal circuitry that is coupled to one or more sensors configured to monitor one or more physiological parameters of the patient. As shown, the sensors can be coupled to the medical device controller 600 via a wired or wireless connection. The sensors can include one or more ECG sensing electrodes 622, and non-ECG physiological sensors 623 such as vibration sensor 624, tissue fluid monitors 626 (for example, based on ultra-wide band RF devices), and motion sensors (for example, accelerometers, gyroscopes, and/or magnetometers). In some implementations, the sensors can include a plurality of conventional ECG sensing electrodes in addition to digital sensing electrodes.

The sensing electrodes 622 can be configured to monitor a patient's ECG information. For example, by design, the digital sensing electrodes 622 can include skin-contacting electrode surfaces that may be deemed polarizable or non-polarizable depending on a variety of factors including the metals and/or coatings used in constructing the electrode surface. All such electrodes can be used with the principles, techniques, devices and systems described herein. For example, the electrode surfaces can be based on stainless steel, noble metals such as platinum, or Ag—AgCl.

In some examples, the electrodes 622 can be used with an electrolytic gel dispersed between the electrode surface and the patient's skin. In certain implementations, the electrodes 622 can be dry electrodes that do not need an electrolytic material. As an example, such a dry electrode can be based on tantalum metal and having a tantalum pentoxide coating as is described above. Such dry electrodes can be more comfortable for long term monitoring applications.

Referring back to FIG. 6, the vibration sensors 624 can be configured to detect cardiac or pulmonary vibration information. For example, the vibration sensors 624 can detect a patient's heart valve vibration information. For example, the vibration sensors 624 can be configured to detect cardio-vibrational signal values including any one or all of S1, S2, S3, and S4. From these cardio-vibrational signal values or heart vibration values, certain heart vibration metrics may be calculated, including any one or more of electromechanical activation time (EMAT), average EMAT, percentage of EMAT (% EMAT), systolic dysfunction index (SDI), and left ventricular systolic time (LVST). The vibration sensors 624 can also be configured to detect heart wall motion, for instance, by placement of the sensor in the region of the apical beat. The vibration sensors 624 can include a vibrational sensor configured to detect vibrations from a patient's cardiac and pulmonary system and provide an output signal responsive to the detected vibrations of a targeted organ, for example, being able to detect vibrations generated in the trachea or lungs due to the flow of air during breathing. In certain implementations, additional physiological information can be determined from pulmonary-vibrational signals such as, for example, lung vibration characteristics based on sounds produced within the lungs (for example, stridor, crackle, and so forth). The vibration sensors 624 can also include a multi-channel accelerometer, for example, a three-channel accelerometer configured to sense movement in each of three orthogonal axes such that patient movement/body position can be detected and correlated to detected cardio-vibrations information. The vibration sensors 624 can transmit information descriptive of the cardio-vibrations information to the sensor interface 612 for subsequent analysis.

The tissue fluid monitors 626 can use RF based techniques to assess fluid levels and accumulation in a patient's body tissue. For example, the tissue fluid monitors 626 can be configured to measure fluid content in the lungs, typically for diagnosis and follow-up of pulmonary edema or lung congestion in heart failure patients. The tissue fluid monitors 626 can include one or more antennas configured to direct RF waves through a patient's tissue and measure output RF signals in response to the waves that have passed through the tissue. In certain implementations, the output RF signals include parameters indicative of a fluid level in the patient's tissue. The tissue fluid monitors 626 can transmit information descriptive of the tissue fluid levels to the sensor interface 612 for subsequent analysis.

As further shown in FIG. 6, the controller 600 can further include an accelerometer interface 630 and a set of accelerometers 632. The accelerometer interface 630 can be operably coupled to each of the accelerometers 632 and configured to receive one or more outputs from the accelerometers. The accelerometer interface 630 can be further configured to condition the output signals by, for example, converting analog accelerometer signals to digital signals (if using an analog accelerometer), filtering the output signals, combining the output signals into a combined directional signal (for example, combining each x-axis signal into a composite x-axis signal, combining each y-axis signal into a composite y-axis signal, and combining each z-axis signal into a composite z-axis signal). In some examples, the accelerometer interface 630 can be configured to filter the signals using a high-pass or band-pass filter to isolate the acceleration of the patient due to movement from the component of the acceleration due to gravity.

Additionally, the accelerometer interface 630 can configure the output for further processing. For example, the accelerometer interface 630 can be configured to arrange the output of an individual accelerometer 632 as a vector expressing the acceleration components of the x-axis, they-axis, and the z-axis as received from each accelerometer. The accelerometer interface 630 can be operably coupled to the processor 618 and configured to transfer the output signals from the accelerometers 632 to the processor for further processing and analysis.

As described above, one or more of the accelerometers 632 (for example, accelerometers 108 as described above) can be integrated into one or more components of a medical device. For example, as shown in FIG. 6, an accelerometer 632 (for example, accelerometer 108c as described above) can be integrated into the controller 600. In some examples, an accelerometer 632 can be integrated into one or more of a therapy electrode 620, a sensing electrode 622, a physiological sensor 623, and into other components of a medical device. When controller 600 is included in an HWD, an accelerometer can be integrated into an adhesive ECG sensing and/or therapy electrode patch.

In certain implementations, the cardiac event detector 616 can be configured to monitor a patient's ECG signal for an occurrence of a cardiac event such as an arrhythmia or other similar cardiac event. The cardiac event detector can be configured to operate in concert with the processor 618 to execute one or more methods that process received ECG signals from, for example, the sensing electrodes 622 and determine the likelihood that a patient is experiencing a cardiac event. The cardiac event detector 616 can be implemented using hardware or a combination of hardware and software. For instance, in some examples, cardiac event detector 616 can be implemented as a software component that is stored within the data storage 604 and executed by the processor 618. In this example, the instructions included in the cardiac event detector 616 can cause the processor 618 to perform one or more methods for analyzing a received ECG signal to determine whether an adverse cardiac event is occurring. In other examples, the cardiac event detector 616 can be an application-specific integrated circuit (ASIC) that is coupled to the processor 618 and configured to monitor ECG signals for adverse cardiac event occurrences. Thus, examples of the cardiac event detector 616 are not limited to a particular hardware or software implementation.

In some implementations, the processor 618 includes one or more processors (or one or more processor cores) that each are configured to perform a series of instructions that result in manipulated data and/or control the operation of the other components of the medical device controller 600. In some implementations, when executing a specific process (for example, cardiac monitoring), the processor 618 can be configured to make specific logic-based determinations based on input data received and be further configured to provide one or more outputs that can be used to control or otherwise inform subsequent processing to be carried out by the processor 618 and/or other processors or circuitry with which processor 618 is communicatively coupled. Thus, the processor 618 reacts to specific input stimulus in a specific way and generates a corresponding output based on that input stimulus. In some example cases, the processor 618 can proceed through a sequence of logical transitions in which various internal register states and/or other bit cell states internal or external to the processor 618 can be set to logic high or logic low. As referred to herein, the processor 618 can be configured to execute a function where software is stored in a data store coupled to the processor 618, the software being configured to cause the processor 618 to proceed through a sequence of various logic decisions that result in the function being executed. The various components that are described herein as being executable by the processor 618 can be implemented in various forms of specialized hardware, software, or a combination thereof. For example, the processor 618 can be a digital signal processor (DSP) such as a 24-bit DSP. The processor 618 can be a multi-core processor, for example, having two or more processing cores. The processor 618 can be an Advanced RISC Machine (ARM) processor such as a 32-bit ARM processor or a 64-bit ARM processor. The processor 618 can execute an embedded operating system, and include services provided by the operating system that can be used for file system manipulation, display and audio generation, basic networking, firewalling, data encryption and communications.

As noted above, an ambulatory medical device such as a WCD can be designed to include a digital front-end where analog signals sensed by skin-contacting electrode surfaces of a set of digital sensing electrodes are converted to digital signals for processing. Typical ambulatory medical devices with analog front-end configurations use circuitry to accommodate a signal from a high source impedance from the sensing electrode (for example, having an internal impedance range from approximately 100 kiloohms to one or more megaohms). This high source impedance signal is processed and transmitted to a monitoring device such as processor 618 of the controller 600 as described above for further processing. In certain implementations, the monitoring device, or another similar processor such as a microprocessor or another dedicated processor operably coupled to the sensing electrodes, can be configured to receive a common noise signal from each of the sensing electrodes, sum the common noise signals, invert the summed common noise signals and feed the inverted signal back into the patient as a driven ground using, for example, a driven right leg circuit to cancel out common mode signals.

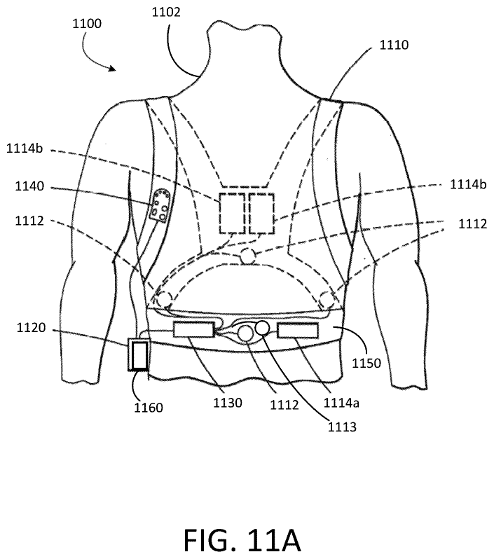

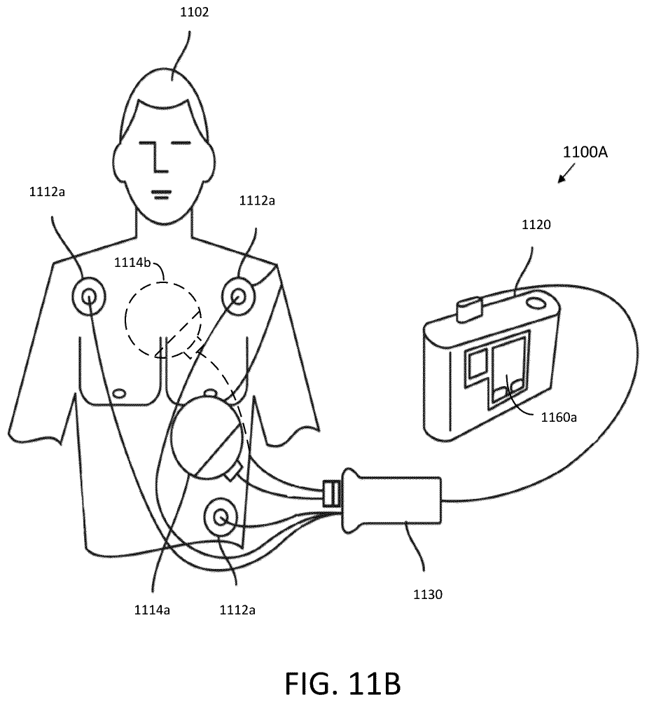

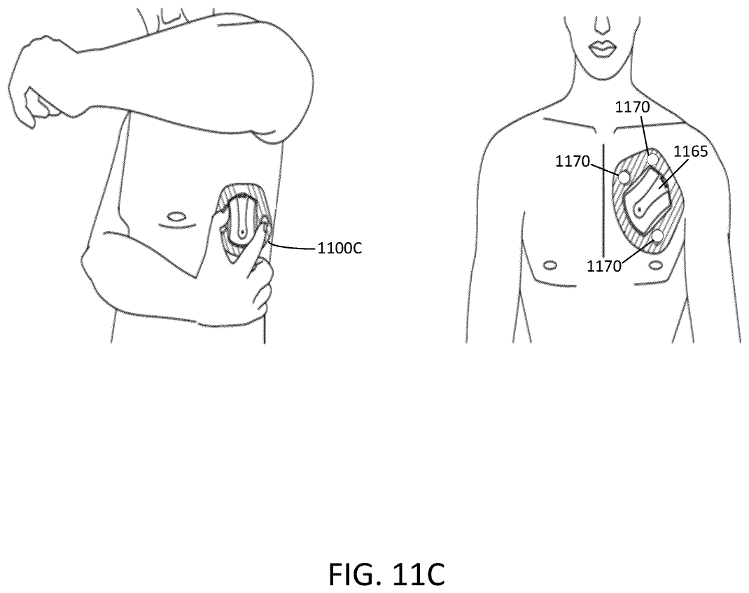

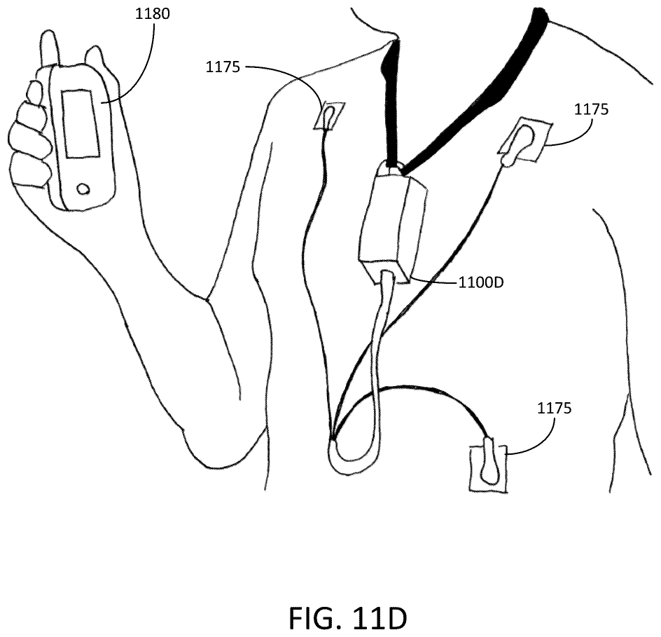

FIG. 11A illustrates an example medical device 1100 that is external, ambulatory, and wearable by a patient 1102, and configured to implement one or more configurations described herein. For example, the medical device 1100 can be a non-invasive medical device configured to be located substantially external to the patient. Such a medical device 1100 can be, for example, an ambulatory medical device that is capable of and designed for moving with the patient as the patient goes about his or her daily routine. For example, the medical device 1100 as described herein can be bodily-attached to the patient such as the LifeVest® wearable cardioverter defibrillator available from ZOLL® Medical Corporation (Chelmsford, MA). Such wearable defibrillators typically are worn nearly continuously or substantially continuously for two to three months at a time. During the period of time in which they are worn by the patient, the wearable defibrillator can be configured to continuously or substantially continuously monitor the vital signs of the patient and, upon determination that treatment is required, can be configured to deliver one or more therapeutic electrical pulses to the patient. For example, such therapeutic shocks can be pacing, defibrillation, or transcutaneous electrical nerve stimulation (TENS) pulses.

The medical device 1100 can include one or more of the following: a garment 1110, one or more ECG sensing electrodes 1112, one or more non-ECG physiological sensors 1113, one or more therapy electrodes 1114a and 1114b (collectively referred to herein as therapy electrodes 1114), a medical device controller 1120 (for example, controller 600 as described above in the discussion of FIG. 6), a connection pod 1130, a patient interface pod 1140, a belt 1150, or any combination of these. In some examples, at least some of the components of the medical device 1100 can be configured to be affixed to the garment 1110 (or in some examples, permanently integrated into the garment 1110), which can be worn about the patient's torso.

The medical device controller 1120 can be operatively coupled to the sensing electrodes 1112, which can be affixed to the garment 1110, for example, assembled into the garment 1110 or removably attached to the garment, for example, using hook and loop fasteners. In some implementations, the sensing electrodes 1112 can be permanently integrated into the garment 1110. The medical device controller 1120 can be operatively coupled to the therapy electrodes 1114. For example, the therapy electrodes 1114 can also be assembled into the garment 1110, or, in some implementations, the therapy electrodes 1114 can be permanently integrated into the garment 1110. In an example, the medical device controller 1120 includes a patient user interface 1160 to allow a patient interface with the externally-worn device. For example, the patient can use the patient user interface 1160 to respond to activity related questions, prompts, and surveys as described herein.

Component configurations other than those shown in FIG. 11A are possible. For example, the sensing electrodes 1112 can be configured to be attached at various positions about the body of the patient 1102. The sensing electrodes 1112 can be operatively coupled to the medical device controller 1120 through the connection pod 1130. In some implementations, the sensing electrodes 1112 can be adhesively attached to the patient 1102. In some implementations, the sensing electrodes 1112 and at least one of the therapy electrodes 1114 can be included on a single integrated patch and adhesively applied to the patient's body.

The sensing electrodes 1112 can be configured to detect one or more cardiac signals. Examples of such signals include ECG signals and/or other sensed cardiac physiological signals from the patient. In certain examples, as described herein, the non-ECG physiological sensors 1113 include components such as one or more of accelerometers, vibrational sensors, RF-based sensors, and other measuring devices for recording additional non-ECG physiological parameters. For example, as described above, the such non-ECG physiological sensors are configured to detect other types of patient physiological parameters and acoustic signals, such as tissue fluid levels, cardio-vibrations, lung vibrations, respiration vibrations, patient movement, and so forth.

In some examples, the therapy electrodes 1114 can also be configured to include sensors configured to detect ECG signals as well as other physiological signals of the patient. The connection pod 1130 can, in some examples, include a signal processor configured to amplify, filter, and digitize these cardiac signals prior to transmitting the cardiac signals to the medical device controller 1120. One or more of the therapy electrodes 1114 can be configured to deliver one or more therapeutic defibrillating shocks to the body of the patient 1102 when the medical device 1100 determines that such treatment is warranted based on the signals detected by the sensing electrodes 1112 and processed by the medical device controller 1120. Example therapy electrodes 1114 can include metal electrodes such as stainless-steel electrodes that include one or more conductive gel deployment devices configured to deliver conductive gel to the metal electrode prior to delivery of a therapeutic shock.

In some implementations, medical devices as described herein can be configured to switch between a therapeutic medical device and a monitoring medical device that is configured to only monitor a patient (for example, not provide or perform any therapeutic functions). For example, therapeutic components such as the therapy electrodes 1114 and associated circuitry can be optionally decoupled from (or coupled to) or switched out of (or switched in to) the medical device. For example, a medical device can have optional therapeutic elements (for example, defibrillation and/or pacing electrodes, components, and associated circuitry) that are configured to operate in a therapeutic mode. The optional therapeutic elements can be physically decoupled from the medical device to convert the therapeutic medical device into a monitoring medical device for a specific use (for example, for operating in a monitoring-only mode) or a patient. Alternatively, the optional therapeutic elements can be deactivated (for example, via a physical or a software switch), essentially rendering the therapeutic medical device as a monitoring medical device for a specific physiologic purpose or a particular patient. As an example of a software switch, an authorized person can access a protected user interface of the medical device and select a preconfigured option or perform some other user action via the user interface to deactivate the therapeutic elements of the medical device.

FIG. 11B illustrates a hospital wearable defibrillator 1100A that is external, ambulatory, and wearable by a patient 1102. Hospital wearable defibrillator 1100A can be configured in some implementations to provide pacing therapy, for example, to treat bradycardia, tachycardia, and asystole conditions. The hospital wearable defibrillator 1100A can include one or more ECG sensing electrodes 1112a, one or more therapy electrodes 1114a and 1114b, a medical device controller 1120 and a connection pod 1130. For example, each of these components can be structured and function as like number components of the medical device 1100. For example, the electrodes 1112a, 1114a, 1114b can include disposable adhesive electrodes. For example, the electrodes can include sensing and therapy components disposed on separate sensing and therapy electrode adhesive patches. In some implementations, both sensing and therapy components can be integrated and disposed on a same electrode adhesive patch that is then attached to the patient. For example, the front adhesively attachable therapy electrode 1114a attaches to the front of the patient's torso to deliver pacing or defibrillating therapy. Similarly, the back adhesively attachable therapy electrode 1114b attaches to the back of the patient's torso. In an example scenario, at least three ECG adhesively attachable sensing electrodes 1112a can be attached to at least above the patient's chest near the right arm, above the patient's chest near the left arm, and towards the bottom of the patient's chest in a manner prescribed by a trained professional.

A patient being monitored by a hospital wearable defibrillator and/or pacing device may be confined to a hospital bed or room for a significant amount of time (for example, 75% or more of the patient's stay in the hospital). As a result, a user interface 1160a can be configured to interact with a user other than the patient, for example, a nurse, for device-related functions such as initial device baselining, setting and adjusting patient parameters, and changing the device batteries.

In some examples, the hospital wearable defibrillator 1100A can further include one or more motion sensors such as accelerometers. For example, an accelerometer can be integrated into one or more of a sensing electrode 1112a (for example, integrated into the same patch as the sensing electrode), a therapy electrode 1114a (for example, integrated into the same patch as the therapy electrode), the medical device controller 1120, the connection pod 1130, and various other components of the hospital wearable defibrillator 1100A.

In some implementations, an example of a therapeutic medical device that includes a digital front-end in accordance with the systems and methods described herein can include a short-term defibrillator and/or pacing device. For example, such a short-term device can be prescribed by a physician for patients presenting with syncope. A wearable defibrillator can be configured to monitor patients presenting with syncope by, for example, analyzing the patient's physiological and cardiac activity for aberrant patterns that can indicate abnormal physiological function. For example, such aberrant patterns can occur prior to, during, or after the onset of syncope. In such an example implementation of the short-term wearable defibrillator, the electrode assembly can be adhesively attached to the patient's skin and have a similar configuration as the hospital wearable defibrillator described above in connection with FIG. 11A.

FIGS. 11C and 11D illustrate example wearable patient monitoring devices with no treatment or therapy functions. For example, such devices are configured to monitor one or more physiological parameters of a patient, for example, for remotely monitoring and/or diagnosing a condition of the patient. For example, such physiological parameters can include a patient's ECG information, tissue (for example, lung) fluid levels, cardio-vibrations (for example, using accelerometers or microphones), and other related cardiac information. A cardiac monitoring device is a portable device that the patient can carry around as he or she goes about their daily routine.

Referring to FIG. 11C, an example wearable patient monitoring device 1100C can include tissue fluid monitors 1165 that use RF based techniques to assess fluid levels and accumulation in a patient's body tissue. Such tissue fluid monitors 1165 can be configured to measure fluid content in the lungs, typically for diagnosis and follow-up of pulmonary edema or lung congestion in heart failure patients. The tissue fluid monitors 1165 can include one or more antennas configured to direct RF waves through a patient's tissue and measure output RF signals in response to the waves that have passed through the tissue. In certain implementations, the output RF signals include parameters indicative of a fluid level in the patient's tissue. In examples, device 1100C may be a cardiac monitoring device that also includes digital sensing electrodes 1170 for sensing ECG activity of the patient. Device 1100C can pre-process the ECG signals via one or more ECG processing and/or conditioning circuits such as an ADC, operational amplifiers, digital filters, signal amplifiers under control of a microprocessor. Device 1100C can transmit information descriptive of the ECG activity and/or tissue fluid levels via a network interface to a remote server for analysis. Additionally, in certain implementations, the device 1100C can include one or more accelerometers for measuring motion signals as described herein.

Referring to FIG. 11D, another example wearable cardiac monitoring device 1100D can be attached to a patient via at least three adhesive digital cardiac sensing electrodes 1175 disposed about the patient's torso. Additionally, in certain implementations, the device 1100D can include one or accelerometers integrated into, for example, one or more of the digital sensing electrodes for measuring motion signals as described herein.

Cardiac devices 1100C and 1100D are used in cardiac monitoring and telemetry and/or continuous cardiac event monitoring applications, for example, in patient populations reporting irregular cardiac symptoms and/or conditions. These devices can transmit information descriptive of the ECG activity and/or tissue fluid levels via a network interface to a remote server for analysis. Example cardiac conditions that can be monitored include atrial fibrillation (AF), bradycardia, tachycardia, atrio-ventricular block, Lown-Ganong-Levine syndrome, atrial flutter, sino-atrial node dysfunction, cerebral ischemia, pause(s), and/or heart palpitations. For example, such patients may be prescribed a cardiac monitoring for an extended period of time, for example, 10 days to 30 days, or more. In some ambulatory cardiac monitoring and/or telemetry applications, a portable cardiac monitoring device can be configured to substantially continuously monitor the patient for a cardiac anomaly, and when such an anomaly is detected, the monitor can automatically send data relating to the anomaly to a remote server. The remote server may be located within a 24-hour manned monitoring center, where the data is interpreted by qualified, cardiac-trained reviewers and/or HCPs, and feedback provided to the patient and/or a designated HCP via detailed periodic or event-triggered reports. In certain cardiac event monitoring applications, the cardiac monitoring device is configured to allow the patient to manually press a button on the cardiac monitoring device to report a symptom. For example, a patient can report symptoms such as a skipped beat, shortness of breath, light headedness, racing heart rate, fatigue, fainting, chest discomfort, weakness, dizziness, and/or giddiness. The cardiac monitoring device can record predetermined physiologic parameters of the patient (for example, ECG information) for a predetermined amount of time (for example, 1 minute to 30 minutes before and 1 minute to 30 minutes after a reported symptom). As noted above, the cardiac monitoring device can be configured to monitor physiologic parameters of the patient other than cardiac related parameters. For example, the cardiac monitoring device can be configured to monitor, for example, cardio-vibrational signals (for example, using accelerometers or microphones), pulmonary-vibrational signals, breath vibrations, sleep related parameters (for example, snoring, sleep apnea), tissue fluids, among others.

In some examples, the devices described herein (for example, FIGS. 11A to 11D) can communicate with a remote server via an intermediary or gateway device 1180 such as that shown in FIG. 11D. For instance, devices such as shown in FIGS. 11A to 11D can be configured to include a network interface communications capability as described herein in reference to, for example, FIG. 6.

Although the subject matter contained herein has been described in detail for the purpose of illustration, it is to be understood that such detail is solely for that purpose and that the present disclosure is not limited to the disclosed embodiments, but, on the contrary, is intended to cover modifications and equivalent arrangements that are within the scope of the appended claims. For example, it is to be understood that the present disclosure contemplates that, to the extent possible, one or more features of any embodiment can be combined with one or more features of any other embodiment.

Other examples are within the scope of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

1. A system for removing device signals from surface electrical signals received from an ambulatory patient worn device, the system comprising:
a wearable cardiac monitoring device configured to be continuously worn by a patient, the wearable cardiac monitoring device comprising
signal generation circuitry configured to provide at least one device-generated signal having a predetermined frequency;
a skin-contacting surface configured to inject the at least one device-generated signal into a skin of the patient;
at least one electrode configured to acquire at least one surface electrical signal from the skin of the patient, the at least one surface electrical signal comprising the at least one device-generated signal and at least one physiological signal generated by the patient; and
processing circuitry different from the signal generation circuitry, the processing circuitry comprising at least one processor configured to
sample the at least one surface electrical signal from the patient,
identify, based on the predetermined frequency, aliasing content generated by the at least one device-generated signal within the at least one surface electrical signal,
filter the aliasing content generated by the at least one device-generated signal to isolate the at least one physiological signal, and
provide the at least one physiological signal.

2. The system of claim 1, wherein the processing circuitry and the signal generation circuitry are asynchronous.

3. The system of claim 1, wherein a phase of the at least one device-generated signal provided by the signal generation circuitry is arbitrary relative to a phase of the at least one device-generated signal within the at least one surface electrical signal.

4. The system of claim 1, wherein the predetermined frequency is 100 Hz, 200 Hz, 300 Hz, 400 Hz, 500 Hz, 1 kHz, 2 kHz, 4 kHz, or 8 kHz.

5. The system of claim 4, wherein:
to sample comprises to sample at a predetermined rate; and
the predetermined frequency is an integer multiple of the predetermined rate.

6. The system of claim 5, wherein the integer multiple is 2, 3, 4, 5, or 6.

7. The system of claim 1, wherein:
to sample comprises to sample at a predetermined rate;
the predetermined frequency is an integer multiple of the predetermined rate; and
the at least one processor is further configured to:
control the signal generation circuitry to vary the predetermined frequency among a range of predetermined frequency values,
determine a quality metric of the at least one surface electrical signal during provision of the at least one device-generated signal at each value of the range of predetermined frequency values, and
identify a predetermined frequency value at which the quality metric meets at least one predetermined criterion.

8. The system of claim 7, wherein the range of predetermined frequency values comprises one or more of 100 Hz, 200 Hz, 300 Hz, 400 Hz, 500 Hz, 1 kHz, 2 kHz, 4 kHz, or 8 kHz.

9. The system of claim 7, wherein the at least one predetermined criterion comprises one or more of a threshold value or a relative quality metric.

10. The system of claim 7, wherein the quality metric comprises one or more of a signal to noise ratio or an electrocardiogram (ECG) recognition metric.

11. The system of claim 1, wherein the at least one processor is further configured to isolate the at least one device-generated signal from the at least one surface electrical signal either periodically or continuously.

12. The system of claim 1, wherein the skin-contacting surface is part of at least one of a therapy electrode, an ECG electrode, a combination therapy and ECG electrode, a dry/capacitive electrode, and a fabric electrode.

13. The system of claim 1, wherein to provide the at least one device-generated signal comprises to modulate, by the signal generation circuitry, a base electrical signal at the predetermined frequency.

14. The system of claim 1, wherein the at least one processor is configured to implement at least a portion of the signal generation circuitry in software.

15. The system of claim 1, wherein:
the at least one electrode comprises a plurality of electrodes;
the at least one surface electrical signal comprises a plurality of surface electrical signals; and
to identify comprises to identify the aliasing content generated by the at least one device-generated signal within each surface electrical signal of the plurality of surface electrical signals.

16. The system of claim 1, further comprising a clock circuit shared by the signal generation circuitry and the at least one processor, the clock circuit configured to provide a common reference point to the signal generation circuitry and the at least one processor.

17. The system of claim 1, further comprising a network interface coupled to the at least one processor, wherein to provide the at least one physiological signal comprises to transmit the at least one physiological signal to a remote server distinct from the wearable cardiac monitoring device via the network interface.

18. The system of claim 1, wherein the at least one physiological signal comprises an ECG signal.

19.-35. (canceled)

36. A method for removing device signals from surface electrical signals received from an ambulatory patient worn device, the method comprising:
generating at least one device-generated electrical signal at a predetermined frequency;
injecting the at least one device-generated electrical signal into a skin of a patient;
sampling at least one surface electrical signal from the patient at a predetermined rate, the at least one surface electrical signal comprising the at least one device-generated electrical signal and at least one physiological signal generated by the patient, wherein a phase of the at least one device-generated electrical signal as included in the at least one surface electrical signal is arbitrary relative to a phase of the at least one device-generated electrical signal as injected into the skin of the patient;
identifying, based on the predetermined frequency, aliasing content generated by the at least one device-generated electrical signal within the at least one surface electrical signal;
filtering the aliasing content generated by the at least one device-generated electrical signal to isolate the at least one physiological signal; and
providing the at least one physiological signal.

37.-39. (canceled)

40. The method of claim 36, wherein generating the at least one device-generated electrical signal comprises generating the at least one device-generated electrical signal at a predetermined frequency that is an integer multiple of the predetermined rate, and wherein the method further comprises:
varying the predetermined frequency among a range of predetermined frequency values;
determining a quality metric of the at least one surface electrical signal during provision of the at least one device-generated electrical signal at each value of the range of predetermined frequency values; and
identifying a predetermined frequency value at which the quality metric meets at least one predetermined criterion.

41.-43. (canceled)

44. The method of claim 36, further comprising isolating the at least one device-generated electrical signal from the at least one surface electrical signal either periodically or continuously.

45.-46. (canceled)

47. The method of claim 36, further comprising generating a clock signal to provide a common reference point for generating the at least one device-generated electrical signal at the predetermined frequency and sampling the at least one surface electrical signal from the patient at the predetermined rate.

48.-49. (canceled)
